SEQUENCE LISTING (5 sequences)

SEQ ID NO: 1 - protein, 529 aa, Unknown
  Derived from uncultured archaeon
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 2 - DNA, 2955 bp, Unknown
  Derived from uncultured archaeon
atggcccgcatcctcgctttcgacatcggaatctctagtatcggatgggccttctctgaaaacgacgaactgaaagactgcggcgtgagaatcttcacaaaggttgaaaaccctaaaacaggcgagtctttagctctgccacgtaggttggcccgctccgcccgaaaaaggctggctcggcggaaggctcgcctcaaccacttgaagcatttgatagctaatgagttcaaactgaactacgaagattaccagtccttcgacgagtcattggcaaaagcctacaaaggcagccttatcagtccttatgagttgagatttcgcgcactcaacgaactgctttctaagcaagactttgctagggtcattctgcacatcgcaaaacggcgaggttatgacgatatcaagaactccgacgataaagaaaagggagccattctcaaggcgatcaaacagaatgaggaaaaattggcaaactaccagagtgtgggcgagtatctgtataaagagtatttccagaagtttaaggaaaacagcaaggagtttacaaacgtcagaaataaaaaggagtcttacgagagatgcatcgcgcagtcattcctcaaagatgagctgaagctgatatttaagaagcaacgcgaatttggtttctcattctctaagaagttcgaagaggaggttctttccgtggcgttttacaagagggcgctcaaagacttctcccacctggttggtaactgtagtttcttcacggatgagaagcgagctcccaaaaattctcccctggctttcatgtttgttgccctgactcggatcattaacctgctgaacaacctgaaaaatactgaagggatcttgtatacgaaggacgacctaaatgcactcctgaatgaagtgctcaaaaacggaactctaacctataaacagaccaagaaattactggggctctctgacgactacgagttcaagggcgagaagggtacttattttatcgaattcaaaaagtataaggagttcattaaagcattgggggaacacaacctcagccaggacgatctcaatgaaattgccaaggacatcacgctgattaaagacgagataaaactgaaaaaggcactggccaagtatgacctcaaccagaaccagatcgactctctgtccaagctggagttcaaagaccacctaaacatatccttcaaagccctgaaactggtcacccctctaatgctcgaaggaaaaaaatacgacgaggcgtgtaatgaactgaatcttaaggtggccatcaatgaggataagaaggactttcttccagcctttaacgagacatattacaaagacgaggtcacaaacccggttgtgctgagggccataaaagagtatcggaaggttctgaatgccctcctgaagaagtacggcaaagtgcacaaaataaatatcgaattggctagggaggtggggaagaaccattctcagcgagcaaagatcgagaaagagcagaatgagaactacaaagccaagaaagacgccgaactggagtgcgaaaagctggggcttaaaataaacagtaaaaacatcctgaaattaagattgttcaaagagcaaaaggagttttgcgcctactcaggggaaaaaatcaaaatatcagacctgcaggacgagaaaatgctggagatcgaccacatctatccgtatagcaggtcatttgacgattcctacatgaacaaagtgcttgtgtttaccaaacagaaccaagaaaagctgaaccaaaccccctttgaggctttcggaaacgactcagccaagtggcagaaaatcgaagtcctagccaagaatctgcctacaaaaaaacaaaagaggattcttgataagaactataaggacaaggaacagaaaaactttaaagacaggaacctgaatgacacgaggtacattgcgcgactggttctaaactataccaaagactacctggatttcctccctctgagcgacgacgagaatactaaactgaatgatacccagaaaggctcaaaggtccacgttgaggctaagtccgggatgctgactagcgccctccgccacacgtggggcttcagcgccaaagatcggaataatcatcttcatcacgctattgatgcagtaatcatagcctacgctaacaacagcatcgtgaaagccttctccgatttcaagaaagaacaggagtctaatagcgccgagttgtacgccaagaaaatttccgaattggactataaaaataagagaaaattcttcgaacccttctccgggtttcgccaaaaggtcttagataagatcgacgagattttcgtttccaagcccgaaagaaaaaagccttcaggggcactgcacgaagagacattccgcaaggaagaggaattttaccaatcttacggtggtaaagagggagttctgaaggctctggagcttgggaagatccgcaaggtaaacgggaaaatcgtgaaaaacggggacatgttcagggtggatatcttcaagcacaaaaagaccaacaagttctacgcagtacccatctacactatggatttcgctttaaaggttctcccaaataaggcggtggctcgatcgaagaaaggagagatcaaggactggatcttaatggatgaaaattacgagttttgcttctcgctctacaaagatagcctgattctgatccagacaaaagacatgcaggaaccagaatttgtttattataacgccttcacgagcagtacagtgtccctgattgtgagcaagcatgataacaagttcgagactctgtctaagaatcagaaaatccttttcaagaacgccaacgagaaggaggtcatcgcaaagtcaattggcatccaaaacctgaaggtgttcgagaaatacatagtgtccgcactcggtgaagtaactaaagccgaatttcgacagcgcgaggattttaagaaataa

SEQ ID NO: 3 - RNA, 182 nt, Artificial Sequence
  WT sgRNA
gguucacugauaaaguggagaaccgcuucaccaaaagcugucccuuaggggauuagaacuugagugaaggugggcugcuugcaucagccuaaugucgagaagugcuuucuucggaaaguaacccucgaaacaaauucauuugaaagaaugaaggaaugcaacggaaauuaggugcgcuuggc

SEQ ID NO: 4 - RNA, 178 nt, Artificial Sequence
  delta AUUU
gguucacugauaaaguggagaaccgcuucaccaaaagcugucccuuaggggauuagaacuugagugaaggugggcugcuugcaucagccuaaugucgagaagugcuuucuucggaaaguaacccucgaaacaaauucgaaagaaugaaggaaugcaacggaaauuaggugcgcuuggc

SEQ ID NO: 5 - RNA, 129 nt, Artificial Sequence
  mutant sgRNA
accgcuucaccgagugaaggugggcugcuugcaucagccuaaugucgagaagugcuuucuucggaaaguaacccucgaaacaaagaaaggaaugcaaccacacacacagugggcuaccuuuuauuuuuu

(19) United States
(12) Patent Application Publication
(10) Pub. No.: US 2025/0340854 A1
(43) Pub. Date: Nov. 6, 2025

(54) ENGINEERED Cas12f PROTEIN

(71) Applicant: THE UNIVERSITY OF TOKYO, Tokyo (JP)

(72) Inventors: Osamu NUREKI, Tokyo (JP); Hiroshi NISHIMASU, Tokyo (JP); Satoru TAKEDA, Tokyo (JP); Tsukasa OHMORI, Shimotsuke-shi (JP); Takafumi HIRAMOTO, Shimotsuke-shi (JP)

(21) Appl. No.: 18/033,009

(22) PCT Filed: Nov. 1, 2021
(86) PCT No.: PCT/JP2021/040281

§ 371 Date: Apr. 20, 2023

Related U.S. Application Data
(60) Provisional application No. 63/107,541, filed on Oct. 30, 2020.

(51) Int. Cl. C12N 9/22; C12N 15/11

(52) U.S. Cl. CPC C12N 9/226; C12N 15/111; C12N 2310/20

(57) ABSTRACT

A protein is provided that consists of a sequence including any one amino acid sequence of the following (a) to (c), forms a homodimer, and forms a complex with a guide RNA:
(a) An amino acid sequence containing at least one substitution of an amino acid residue selected from the group consisting of I118, Y122, I126, and M178 in an amino acid sequence set forth in SEQ ID NO: 1,
(b) An amino acid sequence in which one to several amino acids are deleted, inserted, substituted, or added in a portion other than amino acid positions 118, 122, 126, and 178 of the amino acid sequence represented by (a) above,
(c) An amino acid sequence having 80% or more identity in a portion other than the amino acid positions 118, 122, 126, and 178 of the amino acid sequence represented by (a) above.

### RELATED APPLICATIONS

This application is a 35 U.S.C. § 371 filing of International Patent Application No. PCT/JP2021/040281, filed Nov. 1, 2021, which claims priority to U.S. Provisional Patent Application Ser. No. 63/107,541, filed Oct. 30, 2020, the entire disclosures of which are hereby incorporated herein by reference.

### SEQUENCE LISTING

The instant application contains a Sequence Listing which has been submitted electronically in ASCII format and is hereby incorporated by reference in its entirety. Said ASCII file, created on Oct. 24, 2023, is named 739278_SGT-022US_ST25.txt and is 10,046 bytes in size.

### TECHNICAL FIELD

The present invention relates to an engineered Cas12f protein and use thereof.

### BACKGROUND ART

Bacterial and archaeal CRISPR-Cas systems provide adaptive immunity against foreign nucleic acids and are classified into two classes (Classes 1 and 2) and six types (types I to VI). The Class 2 system includes types II, V, and VI and contains a single multidomain effector Cas protein such as Cas9 (type II) or Cas12 (type V).

Cas9 binds to dual RNA guides (CRISPR RNA [crRNA] and transactivating crRNA [tracrRNA]) or single guide RNA (sgRNA), is complementary to the 20-nt guide segment of the RNA guide, and cleaves a double-stranded DNA (dsDNA) target at a sequence adjacent to the NGG (N is any nucleotide) protospacer-adjacent motif (PAM).

Among the diverse type V Cas12 enzymes, type V-A Cas12a (also known as Cpf1) binds to crRNA and cleaves a dsDNA target at the TTTV (V is A, G, or C) PAM. Cas9 contains two nuclease domains HNH and RuvC, which each cleave the target strand (TS) and the non-target strand (NTS) of the dsDNA targets.

In contrast, a single RuvC nuclease domain of Cas12a cleaves both TS and NTS. Cas9 and Cas12a exhibit potent nuclease activity in eukaryotic cells and thus are widely used as versatile genome engineering tools.

Recent studies have confirmed that the type V-F Cas12f protein is a highly compact RNA-guided DNA endonuclease (see, for example, Non-Patent Document 1).

### CITATION LIST

### Non-Patent Document

### Non-Patent Document 1

Programmed DNA destruction by miniature CRISPR-Cas14 enzymes. Harrington L B, Burstein D, Chen J S, Paez-Espino D, Ma E, Witte I P, Cofsky J C, Kyrpides N C, Banfield J F, Doudna J A. Science. 2018 Nov. 16; 362 (6416): 839-842.

### SUMMARY OF INVENTION

### Technical Problem

The Cas12f enzyme is composed of 400 to 700 amino acid residues, and is much smaller than Cas9 and Cas12 (950 to 1,400 amino acids). Cas12f1 (also known as Cas14a1) derived from hardly culturable archaea is composed of 529 residues and lacks sequence identity with other known proteins, except for the presence of the RuvC domain.

Despite the small size, Cas12f1 associates with dual crRNA: tracrRNA guide and cleaves a dsDNA target having a TTTR (R is A or G) PAM. The guide RNA of Cas12f1 lacks sequence homology with those of other Cas12 enzymes such as Cas12a, Cas12b, and Cas12e. Therefore, the mechanism of action of the miniature type V-F Cas12f nuclease remains enigmatic.

The present invention has been made in consideration of the above circumstances, and an object of the present invention is to provide an engineered Cas12f protein that is capable of being used as a genome editing tool.

### Solution to Problem

That is, the present invention includes the following aspects.

[1] A protein that consists of a sequence including any one amino acid sequence of the following (a) to (c), forms a homodimer, and forms a complex with a guide RNA:
(a) an amino acid sequence containing at least one substitution of an amino acid residue selected from the group consisting of I118, Y122, I126, and M178 in an amino acid sequence set forth in SEQ ID NO: 1,
(b) an amino acid sequence in which one to several amino acids are deleted, inserted, substituted, or added in a portion other than amino acid positions 118, 122, 126, and 178 of the amino acid sequence represented by (a) above,
(c) an amino acid sequence having 80% or more identity in a portion other than the amino acid positions 118, 122, 126, and 178 of the amino acid sequence represented by (a) above.

[2] The protein according to [1], in which the substitution of the amino acid residue in the amino acid sequence represented by (a) above is a substitution with cysteine.

[3] The protein according to [1] or [2], in which the substitution of the amino acid residue in the amino acid sequence represented by (a) above is I118C and/or Y122C.

[4] The protein according to any one of [1] to [3], in which in the amino acid sequences of (a) to (c) above, a substitution of an amino acid residue of A156 and/or Y146 is further contained, and PAM recognition specificity is expanded.

[5] The protein according to [4], in which in the amino acid sequences (a) to (c) above, the substitution of the amino acid residue is A156N.

[6] A protein that consists of a sequence including any one amino acid sequence of the following (d) to (f), forms a homodimer, and forms a complex with a guide RNA:
(d) an amino acid sequence containing a substitution of an amino acid residue of A156 and/or Y146 in an amino acid sequence set forth in SEQ ID NO: 1,
(e) an amino acid sequence in which one to several amino acids are deleted, inserted, substituted, or added in a portion other than amino acid positions 156 and 146 of the amino acid sequence represented by (d) above,
(f) an amino acid sequence having 80% or more identity in a portion other than the amino acid positions 156 and 146 of the amino acid sequence represented by (d) above.

[7] The protein according to [6], in which in the amino acid sequences (d) to (f) above, the substitution of the amino acid residue is A156N.

[8] The protein according to any one of [1] to [7], further containing at least one mutation selected from the group consisting of N133R, E174R, N177R, S187R, N470R, and N483R.

[9] A polynucleotide encoding the protein according to any one of [1] to [8].

[10] A vector containing the polynucleotide according to [9].

[11] A composition containing:
the protein according to any one of [1] to [8], the polynucleotide according to [9], or the vector according to [10]; and
a guide RNA.

[12] A method for editing genome in an isolated cell using the composition according to [11].

[13] A method for site-specifically modifying a target double-stranded polynucleotide in an isolated cell, the method including:
a step of bringing a target double-stranded polynucleotide, the protein according to any one of [1] to [8], and a guide RNA into contact with each other,
in which the protein cleaves the target double-stranded polynucleotide at a cleavage site located upstream of a PAM sequence in the target double-stranded polynucleotide, and
the protein modifies the target double-stranded polynucleotide in a region that is determined by complementary binding of the guide RNA and the target double-stranded polynucleotide.

[14] A method for site-specifically modifying a target double-stranded polynucleotide in an isolated cell, the method including:
a step of bringing a target double-stranded polynucleotide, a complex of the protein according to any one of [1] to [8] and a nucleic acid base converting enzyme, and a guide RNA into contact with each other,
in which the protein specifically binds to the target double-stranded polynucleotide through the guide RNA, where the protein does not cleave the target double-stranded polynucleotide or cleaves only one strand of the target double-stranded polynucleotide, and
the protein modifies the target double-stranded polynucleotide in a region that is determined by complementary binding of the guide RNA and the target double-stranded polynucleotide.

[15] A method for regulating expression of a gene in an isolated cell, the method including:
a step of bringing a target double-stranded polynucleotide associated with the gene, the protein according to any one of [1] to [8], a guide RNA, and an effector molecule into contact with each other,
in which the protein lacks an ability to cleave one or both strands of a target double-stranded polynucleotide, and
the protein specifically binds to the target double-stranded polynucleotide through the guide RNA, and consequently, the effector molecule specifically acts on the target double-stranded polynucleotide to regulate expression of the gene.

### Advantageous Effects of Invention

According to the present invention, it is possible to provide an engineered Cas12f protein that is capable of being used as a genome editing tool.

### BRIEF DESCRIPTION OF DRAWINGS

FIG. 1 (A) shows the domain structure of Cas12f.

FIG. 1 (B) shows images of the overall structure of the Cas12f-sgRNA-target DNA complex.

FIG. 1 (C) shows images of a molecular surface model of the Cas12f dimer. Two Cas12f protomers (Cas12f.1 and Cas12f.2) are shown as the surface model.

FIG. 1 (D) shows Cas12f.1 and Cas12f.2, shown as a surface model and a ribbon model. The guide RNA backbone is shown as a surface model, where the guide segment and the target DNA are omitted.

FIG. 2 (A) shows images of structural comparison of Cas12f with Cas12a, Cas12b, and Cas12e.

FIG. 2 (B) shows images of a zinc binding site in ZF. 1.

FIG. 2 (C) shows images of a zinc binding site in TNB. 1.

FIG. 2 (D) shows a graph of the results of the X-ray fluorescence analysis. X-ray fluorescence spectra were collected from the purified Cas12f and a sample buffer. Kα and Kβ signals of Zn were detected only from the protein sample. Fe and Ni signals originate from the beamline of the optical system.

FIG. 3 (A) shows images of the structure of the Cas12f homodimer. Cas12f.1 and Cas12f.2 are each shown by surface display and cartoon display.

FIG. 3 (B) shows images of the structure of the Cas12f homodimer. Cas12f.1 and Cas12f.2 are each shown by cartoon display and surface display.

FIG. 3 (C) shows an image of the structure of Cas12f.1.

FIG. 3 (D) shows an image of the structure of Cas12f.2.

FIG. 3 (E) is an image in which Cas12f.1 and Cas12f.2 are superimposed based on NTD.

FIG. 3 (F) is an image in which Cas12f.1 and Cas12f.2 are superimposed based on CTD.

FIG. 4 (A) is a schematic view showing an sgRNA and a target DNA. Disordered regions are enclosed in boxes indicated by dashed lines.

FIG. 4 (B) shows images of the structure of the guide RNA backbone.

FIG. 5 (A) is a schematic view showing an sgRNA.

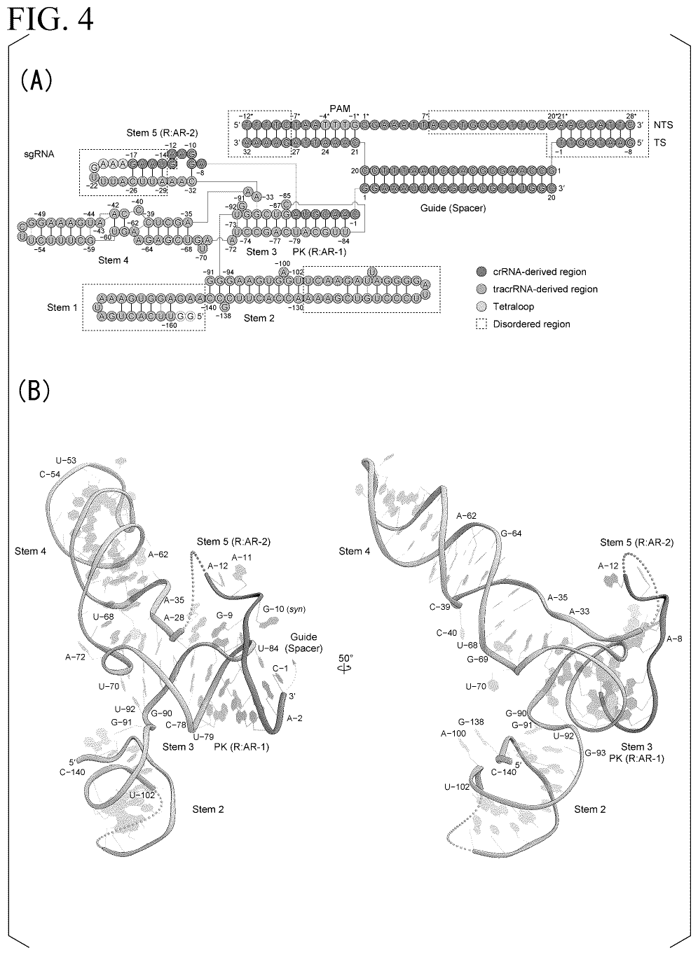

FIG. 4 (B) shows images of the structure of the guide RNA backbone.

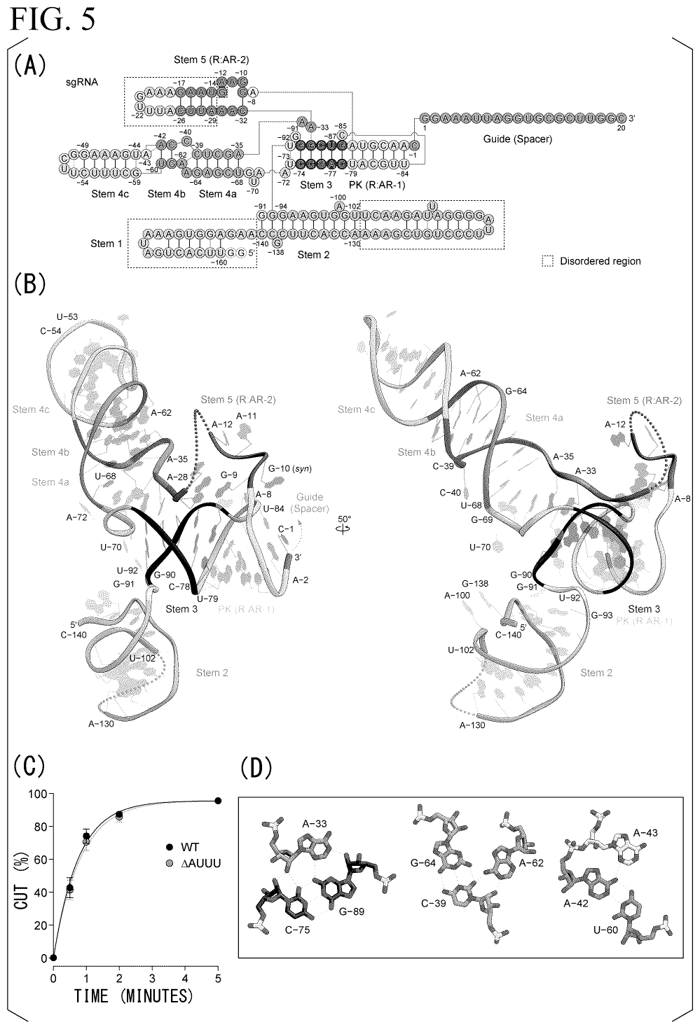

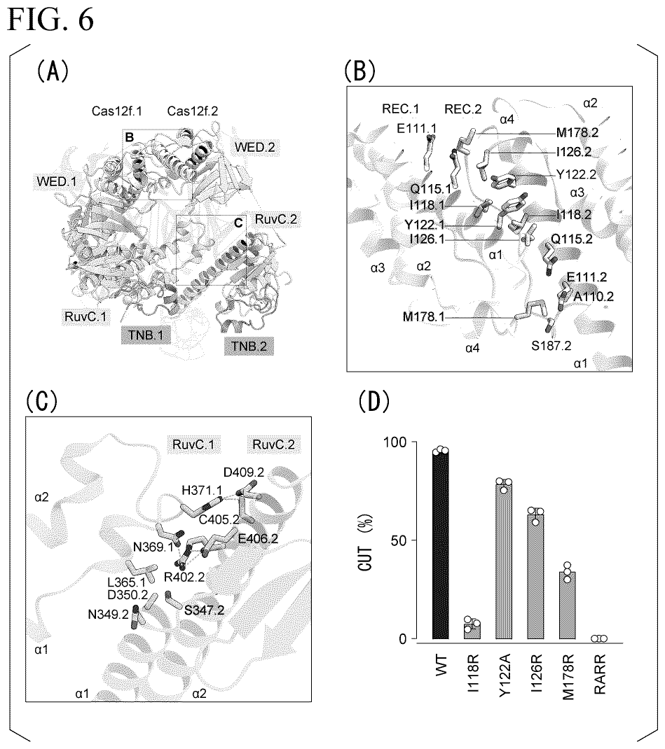

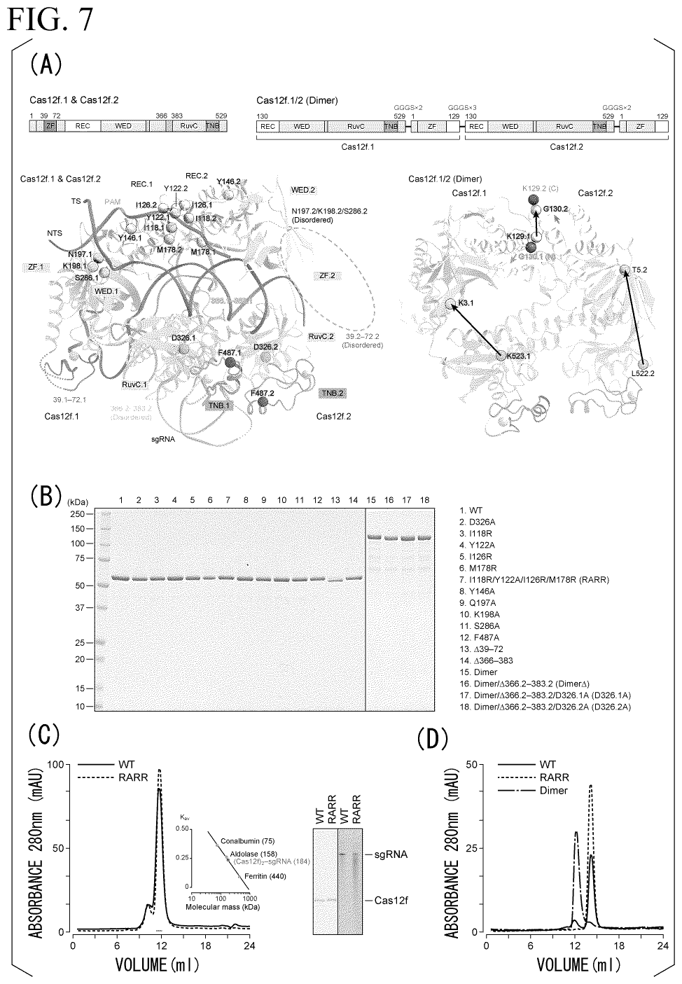

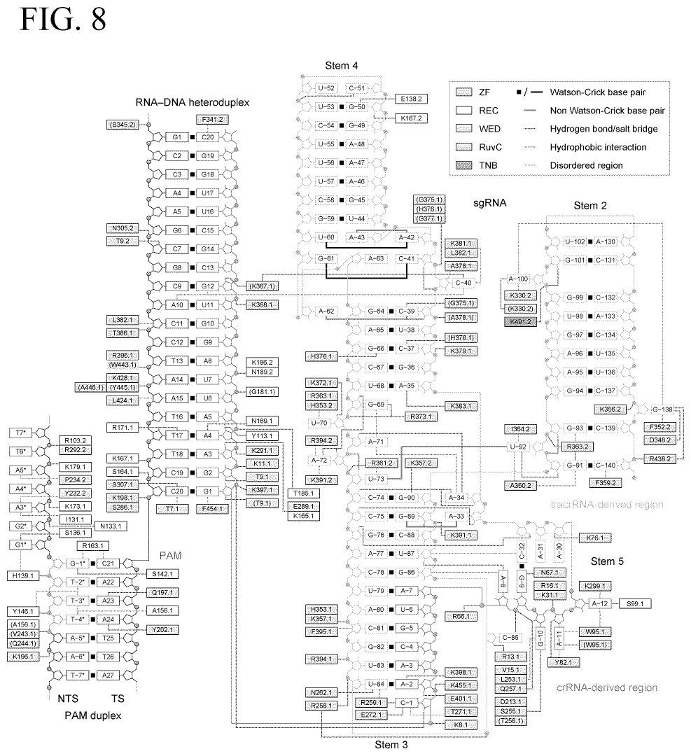

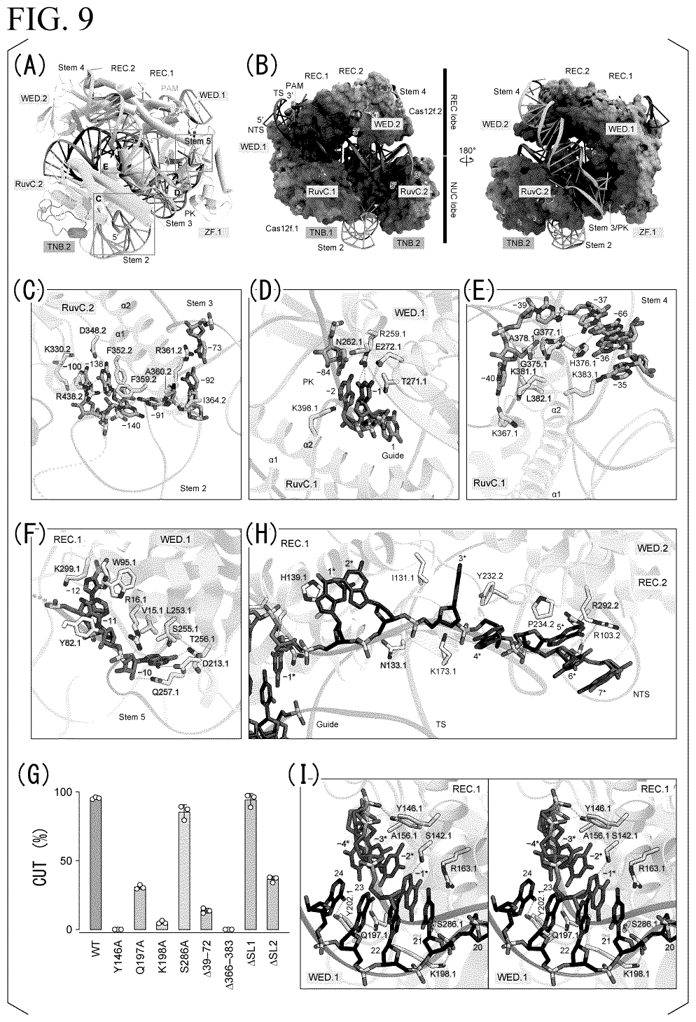

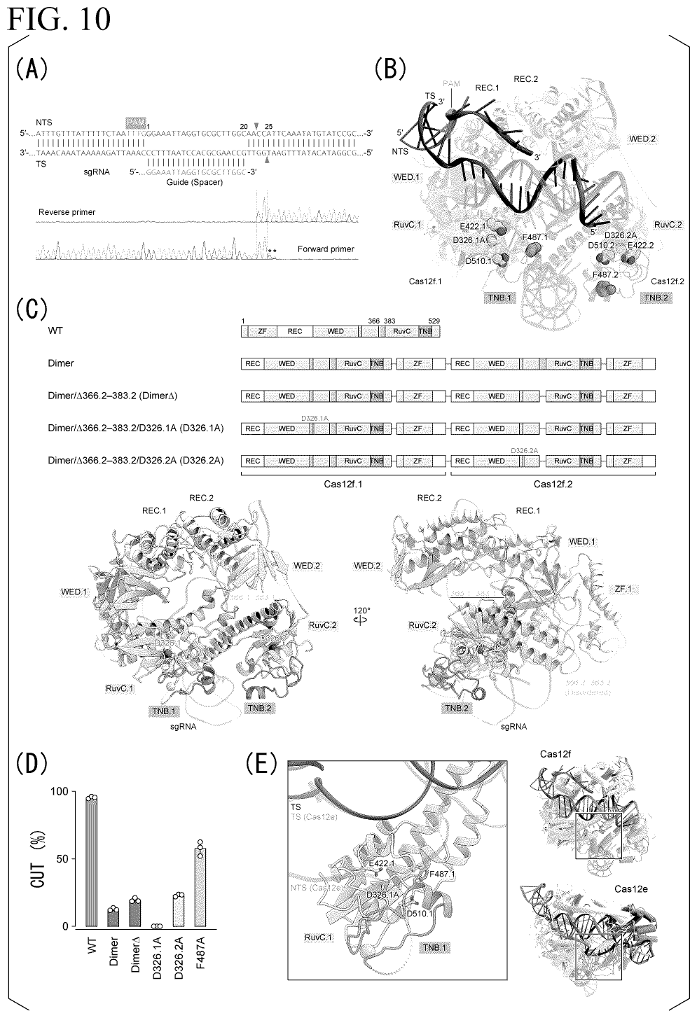

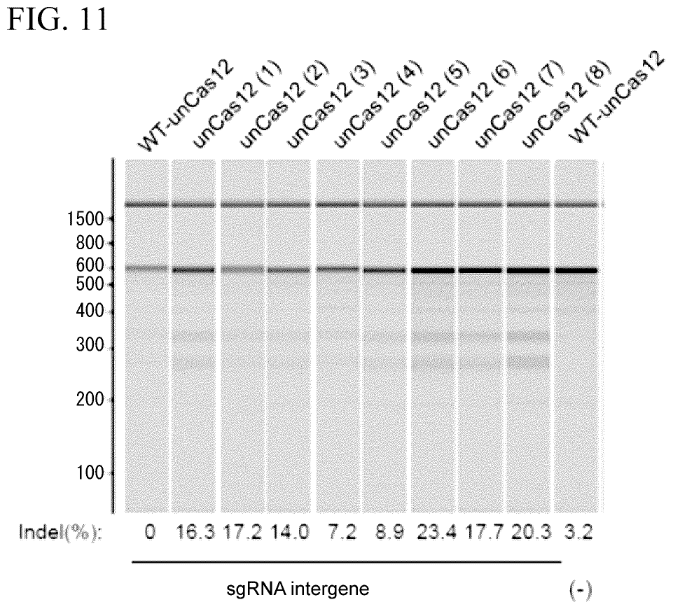

FIG. 5 (C) shows a graph of the results of the cleavage time course obtained from an in vitro DNA cleavage experiment for Cas12f using a WT sgRNA and a ΔAUUU mutant.

FIG. 5 (D) shows images of three bases in the guide RNA backbone.

FIG. 6 (A) shows an image of the dimer interface between Cas12f.1 and Cas12f.2.

FIG. 6 (B) shows an image of the primary interface between REC. 1 and REC. 2.

FIG. 6 (C) shows an image of the secondary interface between REC. 1 and REC. 2.

FIG. 6 (D) shows a graph of the results of the in vitro DNA cleavage activity of WT Cas12f and a dimer interface mutant.

FIG. 7 (A) shows the domain structures of Cas12f mutants. Residues 18 to 93 (ZF) and 366 to 383 (RuvC) in Cas12f.1 are involved in RNA backbone recognition. On the other hand, the corresponding region in Cas12f.2 is exposed to a solvent and disordered. In the case where the N-terminal of Cas12f.2 and the C-terminal of Cas12f.1 are connected with a linker in the dimer mutant, there is a possibility that two molecules of the dimer mutant bind to one sgRNA molecule. To eliminate this possibility, a dimer mutant starting from G130.1 of Cas12f.1 and terminating at K129.1 of Cas12f.2 was prepared using linkers connecting (1) the N-terminal and the C-termini of Cas12f.1 (M1.1 and P529.1), (2) K129.1 of Cas12f.1 and G130.2 of Cas12f.2, and (3) the N-terminal and the C-terminal of Cas12f.2 (M1.2 and P529.2). This design makes it possible to confirm that one dimer mutant molecule binds to one sgRNA molecule.

FIG. 7 (B) shows the results of SDS-PAGE analysis of the WT and mutant Cas12f proteins used in the biochemical experiment.

FIG. 7 (C) shows a graph of the profile results of size exclusion chromatography of the WT Cas12f protein or RARR mutant and the sgRNA. The peak fraction was analyzed by SDS-PAGE and Urea PAGE. The WT Cas12f protein and the RARR mutant were each eluted together with sgRNA at the same position. This indicated that, similar to the WT Cas12f protein, the RARR mutant associates with the sgRNA at least under the conditions tested (20 mM Tris-HCl, pH 8.0, 50 mM NaCl, 5 mM MgCl2, 1 mM DTT).

FIG. 7 (D) shows a graph of the profile results of size exclusion chromatography of the WT Cas12f protein, the RARR mutant, and the dimer mutant. The WT Cas12f protein and the RARR mutant were eluted later than the dimer mutant. This indicated that the WT Cas12f protein and the RARR mutant are present as monomers at least under the conditions tested (20 mM Tris-HCl, pH 8.0, 50 mM NaCl, 5 mM MgCl2, 1 mM DTT).

FIG. 8 is a schematic view regarding nucleic acid recognition.

FIG. 9 (A) shows an image of the recognition site of the guide RNA backbone.

FIG. 9 (B) shows images of the electrostatic surface potential of the Cas12f dimer.

FIG. 9 (C) shows an image of the recognition of the stems 2/3.

FIG. 9 (D) shows an image of the recognition of PK.

FIG. 9 (E) shows an image of the recognition of the stem 4.

FIG. 9 (F) shows an image of the recognition of the stem 5.

FIG. 9 (G) shows a graph of the results of examining the in vitro DNA cleavage activities of the WT Cas12f or Cas12f mutant and the WT sgRNA, and the WT Cas12f and an sgRNA (ΔSL1) in which the stem 1 has been deleted or an sgRNA (ΔSL2) in which the stems 1 and 2 have been deleted. I Data are in terms of mean±SD (n=3).

FIG. 9 (H) shows an image of the recognition of NTS.

FIG. 9 (I) shows images of the recognition of the PAM duplex.

FIG. 10 (A) is a view showing a cleavage site of a target DNA. A plasmid target containing the TTTG PAM was cleaved by the Cas12f-sgRNA complex at 50° C. for min, and the cleavage product was analyzed by Sanger sequencing. The cleavage site is marked with a triangle.

FIG. 10 (B) shows an image of the active sites of Cas12f.1 and Cas12f.2.

FIG. 10 (C) is a view showing the domain structures of D326.1A and D326.2A mutants.

FIG. 10 (D) shows a graph of the results obtained by examining the in vitro DNA cleavage activity of WT Cas12f and a RuvC mutant. Data are in terms of mean±SD (n=3).

FIG. 10 (E), left image shows the active site of Cas12f.1.

FIG. 10 (E), right images show a structural comparison with Cas12e.

FIG. 11 shows the results of indel analysis in cultured cells of the wild-type and mutant Cas12f.

### DESCRIPTION OF EMBODIMENTS

Hereinafter, embodiments of the present invention will be described in detail with reference to the drawings as necessary.

### <<Protein>

The wild-type Cas12f protein is a V-F Cas12f endonuclease consisting of 529 amino acid residues. The full-length amino acid sequence of the wild-type Cas12f protein is set forth in SEQ ID NO:1.

From the crystal structure analysis of the Cas12f protein, the inventors of the present invention revealed that two molecules of Cas12f (referred to as Cas12f.1 and Cas12f.2) form a homodimer and aggregate with one sgRNA molecule to form a complex. Based on the crystal structure analysis data, a region that may be involved in the homodimer formation or interaction with a target DNA was found.

In the present specification, in the case where a base sequence is described, “A” means adenine, “G” means guanine, “C” means cytosine, and “T” means thymine. “R” means adenine or guanine, “Y” means cytosine or thymine, “M” means adenine or cytosine, “H” means adenine, thymine, or cytosine, “V” means adenine, guanine, or cytosine, “D” means adenine, guanine, or thymine, and “N” means adenine, cytosine, thymine, or guanine.

In the present specification, the terms “polypeptide”, “peptide” and “protein” refer to a polymer of amino acid residues and are used interchangeably. In addition, they mean an amino acid polymer in which one or a plurality of amino acids are chemical analogs or modified derivatives of the corresponding naturally occurring amino acids. In the present specification, the single letter and three letter notations for amino acids as defined according to the IUPAC-IUB Joint Commission on Biochemical Nomenclature (JCBN) are used.

In the present specification, in the case where a substitution mutation in an amino acid sequence is indicated, the substitution mutation may be indicated by the one-letter notation of the original amino acid, followed by the position number by a 1- to 4-digit number, and then the one-letter notation of the amino acid with which the original amino acid is substituted. For example, in the case where a mutation in which aspartic acid (D) is substituted with asparagine (N) at amino acid position 1022 occurs, it is denoted as “D1022N”, which has the same meaning as “substitution of Asn with Asp at amino acid position 1022”.

<Cas12f Protein Having a Mutation in at Least One Amino Acid Residue Selected from the Group Consisting of I118, Y122, I126, and M178>

In one embodiment, the present invention provides a protein that consists of a sequence including any one amino acid sequence of the following (a) to (c), forms a homodimer, and forms a complex with a guide RNA.
(a) An amino acid sequence containing at least one substitution of an amino acid residue selected from the group consisting of isoleucine at amino acid position 118, tyrosine at amino acid position 122, isoleucine at amino acid position 126, and methionine at amino acid position 178 in an amino acid sequence set forth in SEQ ID NO: 1
(b) An amino acid sequence in which one to several amino acids are deleted, inserted, substituted, or added in a portion other than amino acid positions 118, 122, 126, and 178 of the amino acid sequence represented by (a) above
(c) An amino acid sequence having 80% or more identity in a portion other than the amino acid positions 118, 122, 126, and 178 of the amino acid sequence represented by (a) above

As will be described later in Examples, Cas12f asymmetrically dimerizes through two interfaces. The primary interface is symmetrical and is formed by hydrophobic residues I118, Y122, I126, and M178. In the case of substituting at least one of these four amino acid residues, it is possible to obtain a Cas12f protein that forms a dimer more tightly.

The amino acid sequence set forth in SEQ ID NO: 1 is the full-length amino acid sequence of the wild-type Cas12f. In (a), the substitution of at least one amino acid residue selected from the group consisting of I118, Y122, I126, and M178 is preferably a substitution with cysteine.

In the substitution of these four amino acid residues, I118C and/or Y122C is more preferable.

In (b), the number of amino acids to be deleted, inserted, substituted, or added is preferably 1 to 105, preferably 1 to 150, more preferably 1 to 79, more preferably 1 to 52, more preferably 1 to 26, still more preferably 1 to 10, and most preferably 1 to 5.

In (c), the identity is preferably 85% or more, more preferably 90% or more, particularly preferably 95% or more, and most preferably 98% or more.

In the present invention, the phrase “forms a homodimer” means that two molecules of the Cas12f monomer dimerize through two interfaces.

In the present invention, the phrase “forms a complex with a guide RNA” means having the ability to bind to a guide RNA. The guide RNA has a sequence complementary to a target DNA at the 5′ terminal thereof and binds to a target DNA through this sequence, whereby the protein according to the present invention is guided to the target DNA.

The protein according to the present embodiment is preferably such a protein that in the amino acid sequences of (a) to (c) above, a substitution of an amino acid residue of A156 and/or Y146 is further contained, and PAM recognition specificity is expanded.

The wild-type Cas12f protein recognizes a PAM sequence of “TTTG”. As will be described later in Examples, the dT (−4*) to dT (−2*) bases of the TTTG PAM form hydrophobic interactions with A156.1 and Y146.1. Therefore, the protein according to the present embodiment is preferably a protein in which the PAM recognition specificity is attenuated by substituting the amino acid residue of A156 and/or Y146. The substituent is preferably asparagine and more preferably contains A156N in the amino acid sequences of (a) to (c).

The protein according to the present embodiment preferably further has at least one mutation selected from the group consisting of N133R, E174R, N177R, S187R, N470R, and N483R. From the results of structural analysis, N133, E174, N177, S187, N470, and N483 are present in the vicinity of the guide RNA, and in the case of being substituted with arginine, the binding between Cas12f and the guide RNA can be reinforced, and the DNA cleavage activity can be improved. That is, the sensitivity of the Cas12f enzyme to the salt concentration can be reduced.

In addition, the protein according to the present embodiment may have nickase activity or may have the inactivated endonuclease activity.

A Cas12f protein having nickase activity or inactivated endonuclease activity is particularly advantageous, for example, in genome editing (single base editing) in which individual bases are modified in terms of the single base unit with high accuracy, or in a method regulating gene expression, as will be described later.

<Cas12f Protein Having a Mutation in Amino Acid Residue A156 and/or Y146>

In one embodiment, the present invention provides a protein that consists of a sequence including any one amino acid sequence of the following (d) to (e), forms a homodimer, and can form a complex with a guide RNA.
(d) An amino acid sequence containing a substitution of an amino acid residue of A156 and/or Y146 in an amino acid sequence set forth in SEQ ID NO: 1
(e) An amino acid sequence in which one to several amino acids are deleted, inserted, substituted, or added in a portion other than amino acid positions 156 and 146 of the amino acid sequence represented by (d) above
(f) An amino acid sequence having 80% or more identity in a portion other than the amino acid positions 156 and 146 of the amino acid sequence represented by (d) above

As described above, the PAM recognition specificity can be attenuated by substituting the amino acid residue of A156 and/or Y146.

In (d), the substitution of A156 and/or Y146 is preferably a substitution with asparagine and more preferably contains A156N.

In (e), the number of amino acids to be deleted, inserted, substituted, or added is preferably 1 to 105, preferably 1 to 150, more preferably 1 to 79, more preferably 1 to 52, more preferably 1 to 26, still more preferably 1 to 10, and most preferably 1 to 5. In (f), the identity is preferably 85% or more, more preferably 90% or more, particularly preferably 95% or more, and most preferably 98% or more.

The protein according to the present embodiment preferably further has at least one mutation selected from the group consisting of N133R, E174R, N177R, S187R, N470R, and N483R. From the results of structural analysis, N133, E174, N177, S187, N470, and N483 are present in the vicinity of the guide RNA, and in the case of being substituted with arginine, the binding between Cas12f and the guide RNA can be reinforced, and the DNA cleavage activity can be improved. That is, the sensitivity of the Cas12f enzyme to the salt concentration can be reduced.

### <<Polynucleotide Encoding Protein>>

In one embodiment, the present invention provides a polynucleotide encoding the Cas12f protein mutant described above.

Examples of such a polynucleotide include a polynucleotide encoding a protein that consists of a sequence including any one base sequence of the following (o1) to (s2), forms a homodimer, and forms a complex with a guide RNA.
(o1) A base sequence in which at least one codon selected from the group consisting of base sequence positions 352 to 354, base sequence positions 364 to 366, base sequence positions 376 to 378, and base sequence positions 532 to 534 of the base sequence set forth in SEQ ID NO: 2 (the base sequence of the wild-type Cas12f) encodes a cysteine
(p1) A base sequence in which one to several bases are deleted, inserted, substituted, or added in a portion other than the base sequence positions 352 to 354, the base sequence positions 364 to 366, the base sequence positions 376 to 378, and the base sequence positions 532 to 534 of the base sequence set forth in SEQ ID NO: 2
(q1) A base sequence in which the identity is 80% or more, preferably 85% or more, more preferably 90% or more, and still more preferably 95% or more in a portion other than the base sequence positions 352 to 354, the base sequence positions 364 to 366, the base sequence positions 376 to 378, and the base sequence positions 532 to 534 of the base sequence set forth in SEQ ID NO: 2
(r1) A base sequence that is capable of hybridizing, under stringent conditions, with DNA consisting of a base sequence complementary to DNA consisting of the base sequence set forth in SEQ ID NO: 2
(s1) A degenerate isomer of the base sequences of (o1) to (r1) Examples of the base sequence encoding cysteine include TGT and TGC.
(o2) A base sequence in which a codon of base sequence positions 436 to 438 and/or base sequence positions 466 to 468 of the base sequence set forth in SEQ ID NO: 2 (the base sequence of the wild-type Cas12f) encodes asparagine
(p2) A base sequence in which one to several bases are deleted, inserted, substituted, or added in a portion other than the base sequence positions 436 to 438 and the base sequence positions 466 to 468 of the base sequence set forth in SEQ ID NO: 2
(q2) A base sequence in which the identity is 80% or more, preferably 85% or more, more preferably 90% or more, and still more preferably 95% or more in a portion other than the base sequence positions 436 to 438 and the base sequence positions 466 to 468 of the base sequence set forth in SEQ ID NO: 2
(r2) A base sequence that is capable of hybridizing, under stringent conditions, with DNA consisting of a base sequence complementary to DNA consisting of the base sequence set forth in SEQ ID NO: 2
(s2) A degenerate isomer of the base sequences of (02) to (r2)

Examples of the base sequence encoding asparagine include AAT and AAC.

In (p1) and (p2), the number of bases that may be deleted, inserted, substituted, or added is preferably 1 to 317, more preferably 1 to 238, still more preferably 1 to 158, particularly preferably 1 to 79, and most preferably 1 to 31.

In (r1) and (r2), examples of the “stringent conditions” include conditions under which hybridization is carried out at 55° C. to 70° C. for several hours or overnight in a hybridization buffer consisting of 5×SSC (composition of 20×SSC: 3 M sodium chloride, 0.3 M citric acid solution, pH 7.0), 0.1% by weight of N-lauroyl sarcosine, 0.02% by weight of SDS, 2% by weight of a blocking reagent for nucleic acid hybridization, and 50% formamide. The washing buffer to be used for washing after incubation is preferably a 1×SSC solution containing 0.1% by weight of SDS and more preferably a 0.1×SSC solution containing 0.1% by weight of SDS.

Multiple codons correspond to one amino acid other than methionine and tryptophan. This is referred to as genetic code degeneracy. In (s1) and (s2), the degenerate isomer of the base sequence means another base sequence corresponding to an amino acid encoded by a certain base sequence.

### <<Vector>

In one embodiment, the present invention provides a vector containing the above-described polynucleotide according to the present invention.

The vector is not particularly limited, and a vector known in the related art, such as a plasmid vector or a virus vector, can be used. Examples of the plasmid vector include a vector having a promoter for expression in animal cells, such as a CAG promoter, an EF1α promoter, an SRα promoter, an SV40 promoter, an LTR promoter, a cytomegalovirus (CMV) promoter, or an HSV-tk promoter.

Examples of the virus vector include a retrovirus vector, an adenovirus vector, an adeno-associated (AAV) vector, a vaccinia virus vector, a lentivirus vector, a herpes virus vector, an alphavirus vector, an EB virus vector, a papilloma virus vector, a foamy virus vector, and a Sindbis virus vector. Since the protein according to the present invention has a small molecular weight, the polynucleotide thereof can be efficiently incorporated into AAV or the like.

In the present embodiment, the base sequence encoding Cas12f may be optimized in terms of the codon, for the expression in a specific cell, such as a eukaryotic cell. Examples of the eukaryotic cell include a human, a mouse, a rat, a rabbit, a dog, a pig, and a non-human primate, but are not limited thereto.

### <<Composition>>

In one embodiment, the present invention provides a composition containing the Cas12f protein mutant described above, a polynucleotide encoding such a protein or a vector containing such a polynucleotide, and a guide RNA.

Since Cas12f contained in the composition according to the present embodiment has a small molecular weight, it is efficiently expressed in vivo. As a result, in the case of using the composition according to the present embodiment, it is possible to easily and rapidly carry out target sequence-specific genome editing and gene expression regulation.

In the present specification, the term “sequence” of “target sequence” means a nucleotide sequence of any length, and includes deoxyribonucleotides or ribonucleotides that are linear, circular, or branched, or single-stranded or double-stranded.

In the present specification, the term “polynucleotide” means a deoxyribonucleotide or ribonucleotide polymer having a linear or circular sequence and which is single-stranded or double-stranded. In addition, the polynucleotide also includes a known analog of a natural nucleotide and a nucleotide modified in at least one of the base portion, the sugar portion, and the phosphate portion (for example, the phosphodiester backbone). In general, an analog of a specific nucleotide has the same base-pairing specificity as the original nucleotide, and thus, for example, an analog of A forms a base pair with T.

In the present specification, the term “guide RNA” means an RNA that mimics the hairpin structure of tracrRNA-crRNA and contains, in the 5′ terminal region, a nucleotide consisting of a base sequence complementary to a target base sequence from 1 base upstream of the PAM sequence in the target double-stranded polynucleotide, to preferably 20 bases or more and 24 bases or less and more preferably 22 bases or more and 24 bases or less. Furthermore, it may include one or more polynucleotides consisting of a base sequence that is non-complementary to the target double-stranded polynucleotide, and consisting of a base sequence that is aligned symmetrically with a single point as an axis to form a complementary sequence and that can form a hairpin structure.

The protein and the guide RNA can be mixed in vitro and in vivo under mild conditions to form a protein-RNA complex. Mild conditions refer to conditions in which the temperature and pH are such that the protein is not decomposed or denatured, where the temperature is preferably 4° C. or higher and 40° C. or lower, and the pH is preferably 4 or more and 10 or less.

In the present embodiment, in the case where the composition contains a gene encoding a modified Cas12f, the gene may be provided as a linear (straightly linear) gene fragment or may be provided in a state incorporated into a vector. In the case where the gene encoding a modified Cas12f is incorporated into a vector, the gene encoding Cas12f and the gene encoding the guide RNA may be provided as the same vector or may be provided as a plurality of individual vectors.

The composition according to the present embodiment is preferably for pharmaceutical use, and more preferably contains a pharmaceutically acceptable carrier. The pharmaceutical composition according to the present embodiment can be administered, for example, orally in the form of a tablet, a coated tablet, a pill, a powder, a granule, a capsule, a liquid, a suspension, or an emulsion, or parenterally in the form of an injection agent, a suppository, or a skin external agent.

As the pharmaceutically acceptable carrier, a carrier used to prepare a general pharmaceutical composition can be used without particular limitation. More specific examples thereof include binders such as gelatin, cornstarch, gum tragacanth, and gum arabic: excipients such as starch and crystalline cellulose: swelling agents such as alginic acid: solvents for an injection agent such as water, ethanol, and glycerin; and adhesives such as rubber-based adhesive and silicone-based adhesive. One kind of pharmaceutically acceptable carrier can be used singly, or two or more kinds thereof can be mixedly used.

The composition according to the present embodiment may further contain additives. Examples of the additive include lubricants such as calcium stearate and magnesium stearate: sweetening agents such as sucrose, lactose, saccharin, and maltitol: flavoring agents such as peppermint and Akamono (Japanese azalea) oil; stabilizers such as benzyl alcohol and phenol: buffering agents such as a phosphoric acid salt and sodium acetate:dissolution assisting agents such as benzyl benzoate and benzyl alcohol; antioxidants; and preservatives. The additive can be used alone, or two or more thereof can be mixed and used.

The composition according to the present embodiments is used to cure and/or prevent one or more diseases or symptoms. The disease or symptoms may be a genetic disease or symptoms resulting from a genetic abnormality.

### <<Method for Site-Specifically Cleaving Target Double-Stranded Polynucleotide>>

In one embodiment, the present invention provides a method for site-specifically modifying a target double-stranded polynucleotide in an isolated cell, the method including a step of bringing a target double-stranded polynucleotide, the Cas protein of the process, and a guide RNA into contact with each other, where the protein cleaves the target double-stranded polynucleotide at a cleavage site located upstream of a PAM sequence in the target double-stranded polynucleotide. Such a method is preferably for site-specifically cleaving a target double-stranded polynucleotide in an isolated cell.

According to the method of the present embodiment, it is possible to easily and rapidly carry out site-specific cleavage of a target double-stranded polynucleotide.

The method for site-specifically cleaving a target double-stranded polynucleotide according to the present embodiment will be described in detail below.

First, the Cas12f protein according to the present embodiment and a guide RNA are brought into contact with each other. The contacting step may be carried out, for example, by mixing the Cas12f protein and the guide RNA under mild conditions and incubating.

Mild conditions refer to conditions in which the temperature and pH are such that the protein is not decomposed or denatured, where the temperature is preferably 4° C. or higher and 40° C. or lower, and the pH is preferably 4 or more and 10 or less. The incubation time is preferably 0.5 hours or more and 1 hour or less. The complex of the Cas12f protein and the guide RNA is stable and thus can remain stable even after being allowed to stand at room temperature for several hours.

The Cas12f protein used in the present embodiment has nuclease activity.

In the present embodiment, the target double-stranded polynucleotide is preferably a sequence including a PAM sequence of “TTTG” in the 5′ to 3′ direction.

Next, the protein and the guide RNA form a complex on the target double-stranded polynucleotide. The protein recognizes the PAM sequence of “TTTG” and cleaves the target double-stranded polynucleotide at a cleavage site located upstream of the PAM sequence.

More specifically, the Cas12f protein recognizes the PAM sequence, the double helix structure of the target double-stranded polynucleotide becomes unduplexed, which starts from the PAM sequence, and the resultant strand anneals with a base sequence complementary to the target double-stranded polynucleotide in the guide RNA, whereby the double helix structure of the target double-stranded polynucleotide is partially unraveled. At this time, the Cas12f protein cleaves a phosphodiester bond of the target double-stranded polynucleotide at a cleavage site located upstream of a PAM sequence in the target double-stranded polynucleotide.

The method according to the present embodiment can be carried out in any environment, in vivo or in vitro. In one embodiment, the method according to the present embodiments can be carried out outside of a living body, that is, ex vivo or in vitro.

### <<First Method for Site-Specifically Modifying Target Double-Stranded Nucleotide>>

In one embodiment, the present invention provides a method for site-specifically modifying a target double-stranded polynucleotide, the method including a step of bringing a target double-stranded polynucleotide, the Cas protein according to the present invention, and a guide RNA into contact with each other, where the protein cleaves the target double-stranded polynucleotide at a cleavage site located upstream of a PAM sequence in the target double-stranded polynucleotide, and the target double-stranded polynucleotide is modified in a region that is determined by the complementary binding of the guide RNA and the target double-stranded polynucleotide.

Such a method is preferably for site-specifically modifying a target double-stranded polynucleotide in an isolated cell.

According to the present embodiment, it is possible to easily and rapidly carry out site-specific modification of a target double-stranded polynucleotide.

The method for site-specifically modifying a target double-stranded nucleotide according to the present embodiment will be described in detail below.

The step of bringing a target double-stranded polynucleotide, the Cas protein, and a guide RNA into contact with each other can be carried out in the same manner as in <Method for site-specifically cleaving target double-stranded polynucleotide> described above.

The target double-stranded polynucleotide, the Cas12f protein, and the guide RNA used in the present embodiment are as described above.

The method for site-specifically modifying a target double-stranded polynucleotide will be described in detail below. The steps up to the site-specific cleavage of the target double-stranded polynucleotide are as described above. Subsequently, it is possible to obtain a target double-stranded polynucleotide modified, according to the intended purpose, in a region that is determined by the complementary binding of the guide RNA and the double-stranded polynucleotide.

In the present specification, the term “modification” means changing the base sequence of the target double-stranded polynucleotide. For example, it includes cleavage of the target double-stranded polynucleotide, a change in the base sequence of the target double-stranded polynucleotide due to the insertion of an exogenous sequence after cleavage (the physical insertion or the insertion due to the replication through homology-directed repair), and a change in the base sequence of the target double-stranded polynucleotide due to non-homologous end joining (NHEJ: rejoining of DNA terminals generated by cleavage). The modification of the target double-stranded polynucleotide in the present embodiment makes it possible to introduce a mutation into the target double-stranded polynucleotide or disrupt the function of the target double-stranded polynucleotide.

The method according to the present embodiment can be carried out in any environment, in vivo or in vitro. In one embodiment, the method according to the present embodiments can be carried out outside of a living body, that is, ex vivo or in vitro.

### <<Second Method for Site-Specifically Modifying Target Double-Stranded Nucleotide>>

In one embodiment, the present invention provides a method for site-specifically modifying a target double-stranded polynucleotide, the method including a step of bringing a target double-stranded polynucleotide, a complex of the Cas protein according to the present invention and a nucleic acid base converting enzyme, and a guide RNA into contact with each other, in which the protein specifically binds to the target double-stranded polynucleotide through the guide RNA, where the protein does not cleave the target double-stranded polynucleotide or cleaves only one strand of the target double-stranded polynucleotide, and the target double-stranded polynucleotide is modified in a region that is determined by complementary binding of the guide RNA and the target double-stranded polynucleotide.

Such a method is preferably for site-specifically modifying a target double-stranded polynucleotide in an isolated cell.

According to the present embodiment, since the Cas12f protein that can tightly bind to a target polynucleotide by forming a dimer is used, it is possible to efficiently carry out precise modification of a target double-stranded polynucleotide site-specifically in terms of the single base unit.

The step of bringing a target double-stranded polynucleotide, a complex of the Cas protein and a nucleic acid base converting enzyme, and a guide RNA into contact with each other can be carried out in the same manner as in <Method for site-specifically cleaving target double-stranded polynucleotide> described above.

The target double-stranded polynucleotide and the guide RNA are as described above. The Cas12f protein used in the present embodiment is a variant that lacks the ability to cleave one or both strands of a target double-stranded polynucleotide, where the variant is described in <DNA cleavage activity of Cas12f protein> above.

The method for site-specifically and precisely modifying a target double-stranded polynucleotide in terms of the single base unit will be described in detail below. In the case where the constitutional components are brought into contact with each other, the Cas12f protein and the guide RNA form a complex and bind to a target double-stranded polynucleotide. Here, the Cas12f protein modifies the base sequence in the target polynucleotide without cleaving the target double-stranded polynucleotide or cleaving only one strand, that is, without causing double-stranded cleavage. The term “modification” is as defined above. In the present embodiment, the modification is preferably carried out in terms of a single base unit, meaning, for example, changing a C-G base pair to a T-A base pair or vice versa.

In the present embodiment, the specific and precise modification (single base editing) in terms of the single base unit is preferably carried out using a nucleic acid base converting enzyme in the complex. The nucleic acid base converting enzyme includes deaminase (a deaminating enzyme). As the deaminase, it is possible to use, for example, cytosine deaminase, cytidine deaminase, or adenosine deaminase. The complex in the present embodiment may contain, in addition to such a nucleic acid base converting enzyme, an Indel formation inhibitor such as a uracil DNA glycosylase inhibitor (UGI) in order to inhibit Indel formation.

The method according to the present embodiment can be carried out in any environment, in vivo or in vitro. In one embodiment, the method can be carried out outside of a living body, that is, ex vivo or in vitro.

### <<Method for Regulating Gene Expression>>

In one embodiment, the present invention provides a method for regulating the expression of a gene, the method including a step of bringing a target double-stranded polynucleotide associated with the gene, the Cas protein according to the present invention, a guide RNA, and an effector molecule into contact with each other, in which the Cas protein specifically binds to the target double-stranded polynucleotide through the guide RNA, and consequently, the effector molecule specifically acts on the target double-stranded polynucleotide to regulate expression of the gene. Such a method is preferably for regulating gene expression in isolated cells.

According to the present embodiment, since the Cas12f protein that can tightly bind to a target polynucleotide by forming a dimer is used, it is possible to efficiently regulate gene expression.

In the present specification, the term “expression” means a process in which a polynucleotide is transcribed into mRNA and/or a process in which the transcribed mRNA is translated into a peptide, a polypeptide, or a protein. In the case where the polynucleotide is derived from genomic DNA, the expression may include the splicing of mRNA in eukaryotic cells.

In the present specification, the term “gene expression” means the conversion of the information contained in a gene into a gene product. The gene product may be a direct transcription product of a gene (for example, an mRNA, a tRNA, an rRNA, an antisense RNA, a ribozyme, an shRNA, a microRNA, a structural RNA, or any other type of RNA) or a protein produced by translation of mRNA. The gene product also includes RNAs modified by processes such as capping, polyadenylation, methylation, and editing, as well as proteins modified by, for example, methylation, acetylation, phosphorylation, ubiquitination, ADP-ribosylation, myristylation, and glycosylation.

In the present specification, the term “regulation” of gene expression means a change in the activity of a gene. The regulation of expression is, for example, activation or repression of a gene, and more particularly activation or repression of transcription, but is not limited thereto.

The method for regulating gene expression using the Cas12f protein according to the present embodiment will be described in detail below.

The step of bringing a target double-stranded polynucleotide, the Cas protein, a guide RNA and an effector molecule into contact with each other can be carried out in the same manner as in <Method for site-specifically cleaving target double-stranded polynucleotide> described above.

The target double-stranded polynucleotide and the guide RNA are as described above. The Cas12f protein used in the present embodiment is a variant that lacks the ability to cleave one or both, preferably both strands of a target double-stranded polynucleotide, where the variant is described in <DNA cleavage activity of Cas12f protein> above.

In the present specification, the term “effector molecule” means a molecule such as a protein or protein domain which is capable of exhibiting a localized effect in cells. The effector molecule can take a variety of different forms, including those that selectively bind to a protein or DNA, for example, in order to regulate biological activity.

The actions of the effector molecule include increasing or decreasing nuclease activity or enzymatic activity, increasing or decreasing gene expression, affecting cell signaling, and the like, but are not limited thereto. Specific examples of the effector molecule that can be used in the present invention include a transcriptional activator or domain such as VP64 or NF-κB p65, a transcriptional repressor or domain such as KRAP, an ERF repressor domain (ERD), or an mSin3A interaction domain (SID), and a chromatin remodeling factor such as DNA methyltransferase, DNA demethylase, a histone acetyltransferase, or a histone deacetylase, but are not limited thereto.

In the present embodiment, the effector molecule is guided to a target double-stranded polynucleotide in the case where a complex of the Cas protein and the guide RNA specifically binds to the target double-stranded polynucleotide. Preferably, the effector molecule is operably linked to the Cas12f protein through a linker depending on the situation.

In the present embodiment, the effector molecule regulates the expression of a gene associated with a target double-stranded polynucleotide by specifically acting on the target double-stranded polynucleotide. As a double-stranded polynucleotide to be targeted, a polynucleotide of a base sequence of a gene of which expression is to be regulated may be selected, or it is also possible to select, for example, a polynucleotide of an upstream base sequence of a gene of which expression is to be regulated, where the base sequence positively or negatively controls directly or indirectly the expression of the gene.

The method according to the present embodiment can be carried out in any environment, in vivo or in vitro. In one embodiment, the method can be carried out outside of a living body, that is, ex vivo or in vitro.

### <<Genome Editing and Gene Therapy Method>>

In one embodiment, the present invention provides a method for carrying out genome editing using the protein or composition described above. In contrast to previously known methods of targeted genetic recombination, the present invention can be carried out efficiently and inexpensively and is applicable to any cell or organism. Any segment of a double-stranded nucleic acid of a cell or organism can be modified by the method according to the present invention. This method uses both the homologous recombination process and the non-homologous recombination process, which are endogenous in all cells.

In one embodiment, the present invention provides a gene therapy method that includes administering to a subject a pharmaceutical composition containing a modified Cas12f protein, a gene encoding the protein or a vector containing the gene, and a guide RNA.

The administration method for the pharmaceutical composition in the present embodiment is not particularly limited and may be appropriately determined depending on the symptoms, body weight, age, sex, and the like of a patient. For example, a tablet, a coated tablet, a pill, a powder, a granule, a capsule, a liquid, a suspension, or an emulsion is administered orally. In addition, an injection agent is intravenously administered singly or as a mixture with a general replacement fluid such as glucose or amino acids, and as necessary, is administered intraarterially, intramuscularly, intracutaneously, subcutaneously, or intraperitoneally.

The dose of the pharmaceutical composition in the present embodiment varies depending on the symptoms, body weight, age, gender, and the like of a human patient or an animal patient and thus cannot be unconditionally determined. However, in the case of oral administration, for example, 1 μg to 10 g per day, for example, 0.01 to 2,000 mg per day in terms of the active ingredient may be administered. In addition, in the case of an injection agent, for example, 0.1 μg to 1 g per day, for example, 0.001 to 200 mg per day in terms of active ingredient may be administered.

In the present specification, the term “genome editing” refers to a new genetic modification technique in which specific gene disruption, knock-in of a reporter gene, or the like is carried out by carrying out targeted genetic recombination or targeted mutation by a technique such as the CRISPR/Cas system. In the present embodiment, the mutation is caused by partial or whole deletion or substitution, or insertion of any sequence, in a target genomic DNA or an expression regulation region of the target genomic DNA.

In addition, in one embodiment, the present invention provides a method of carrying out a targeted DNA insertion or a targeted DNA deletion. This method includes a step of transforming a cell using a nucleic acid construct containing a donor DNA. The schemes regarding DNA insertion and DNA deletion after target gene cleavage can be determined by those skilled in the art according to a known method.

In addition, in one embodiment, the present invention provides genetic manipulation at a specific locus, which is used in both a somatic cell and a germline cell.

In addition, in one embodiment, the present invention provides a method for disrupting a gene in a somatic cell. Here, the gene overexpresses a product that is harmful to a cell or an organism and expresses a product that is harmful to a cell or an organism. Such a gene may be overexpressed in one or more cell types that are generated due to disease. The disruption of the overexpressed gene according to the method of the present invention can result in better health in an individual suffering from a disease caused by the overexpressed gene. That is, gene disruption in only a small proportion of cells can act to reduce the expression level and exhibit a therapeutic effect.

In addition, in one embodiment, the present invention provides a method for disrupting a gene in a germ cell. A cell in which a specific gene is disrupted can be selected to produce an organism that lacks the function of the specific gene. A gene can be completely knocked out in a cell in which the gene is disrupted. The lack of functions in this specific cell may have a therapeutic effect.

In addition, in one embodiment, the present invention further provides the insertion of a donor DNA encoding a gene product. This gene product has a therapeutic effect in the case of being constitutively expressed. For example, a method of inserting a donor DNA encoding an active promoter and an insulin gene into an individual suffering from diabetes in order to cause the insertion of the donor DNA in a population of pancreatic cells. Next, the population of pancreatic cells containing the exogenous DNA produces insulin, whereby the diabetes of the patient can be cured. Further, the donor DNA can be inserted into a crop plant to cause the production of the pharmacologically relevant gene product. A gene of a protein product (for example, insulin, lipase, or hemoglobin) can be inserted into a plant together with a control element (a constitutively active promoter or an inducible promoter) to produce a large amount of a pharmaceutical drug in the plant.

Next, such a protein product can be isolated from the plant. A transgenic plant or a transgenic animal can be produced by a method using a nucleic acid transfer technique. A tissue-type specific vector or a cell-type specific vector can be used to provide gene expression only in selected cells.

Alternatively, the above method can be used in germ cells, whereby it is possible to select cells in which an insertion occurs in a planned manner and a designed genetic alteration is provided through all subsequent cell divisions.

The method according to the present invention can be applied to all organisms, or in cultured cells, cultured tissues, or cultured nuclei (including cells, tissues, or nuclei, which can be used to regenerate an intact organism), or a gamete (for example, eggs or sperm at various stages of their development). The method according to the present invention can be applied to cells derived from any organism (including an insect, a fungus, a rodent, cattle, a sheep, a goat, a chicken, and other animals having agricultural importance, as well as other mammals (including but not limited to a dog, a cat, and a human).

Further, the composition and method according to the present invention can be used in plants. The composition and the method can be used in any of a variety of plant species (for example, monocotyledonous or dicotyledonous plants).

### EXAMPLES

Hereinafter, the present invention will be described in more detail with reference to Examples below. However, the present invention is not limited to these Examples.

### Example 1

A gene (SEQ ID NO: 2) encoding Cas12f (Cas12f1 derived from hardly culturable archaea, also known as Cas14a, 529 amino acid residues) was incorporated into a modified pE-SUMO vector (LifeSensors, Inc.) lacking the SUMO coding region. The N-terminal of the Cas12f to be expressed from the completed construct is designed to have 6 consecutive histidine residues.

An Escherichia coli Rosetta2 (DE3) strain was transformed with the prepared vector. Thereafter, it was cultured in LB medium. At a timing when culturing was carried out until OD=0.8, isopropyl β-D-1-thiogalactopyranoside (IPTG) (final concentration: 0.1 mM) was added thereto as an expression inducer, followed by overnight culturing at 20° C. After culturing, E. coli was recovered by centrifugation.

The recovered cells were suspended in a buffering agent A (20 mM Tris-HCl, pH 8.0, 20 mM imidazole, 1 M NaCl, 1 mM DTT) and subjected to disruption by sonication. The supernatant was recovered by centrifugation (25,000 g, 30 minutes) and mixed with a Ni-NTA Superflow resin (QIAGEN) equilibrated with the buffering agent A, and this mixture was applied onto a Poly-Prep column (Bio-Rad Laboratories Inc.).

A protein of interest was eluted with a buffering agent B (20 mM Tris-HCl, pH 8.0, 0.3 M imidazole, 0.3 M NaCl, 1 mM DTT). This protein was charged onto a HiTrap Heparin HP column (GE Healthcare) equilibrated with buffer solution C (20 mM

Tris-HCl, pH 8.0, 0.3 M NaCl, 1 mM DTT). A protein was eluted with a linear gradient from 0.3 to 2 M NaCl and stored at −80° C. until use.

An sgRNA (SEQ ID NO:3: 5°-(GG)UUCACUGAUAAAGUGGAGAACCGCUUCACCAAAAGCUGUCCCUUAGG GGAUUAGAACUUGAGUGAAGGUGGGCUGCUUGCAUCAGCCUAAUGUCGAG AAGUGCUUUCUUCGGAAAGUAACCCUCGAAACAAAUUCAUUUGAAAGAAU GAAGGAAUGCAACGGAAAUUAGGUGCGCUUGGC-3′) was in vitro transcribed using T7 RNA polymerase and purified by 7M Urea denaturing 10% PAGE.

A Cas12f-sgRNA-target DNA complex was reconstituted by mixing the purified Cas12f D326A mutant, an sgRNA of 180 bases (5′-GG was added to the 180 bases for in vitro transcription), a target DNA strand of 40 bases (manufactured by Sigma-Aldrich Co., LLC), and a non-target DNA strand of 40 bases (manufactured by Sigma-Aldrich Co., LLC), at a molecular ratio of 1:1.2:1.5:1.5.

The Cas12f-sgRNA-target DNA complex was purified by size exclusion chromatography using a Superdex200 Increase 10/300 column equilibrated with a buffering agent D (20 mM Tris-HCl, pH 8.0, 50 mM NaCl, 5 mM MgCl2, 1 mM DTT). The purified complex solution (˜3 mg/mL, 5.4 μL) was mixed with 0.6 μL of ZnCl2 (final concentration: 10 μM), and then a sample (3 μL) was applied in a Cu/Rh 300 mesh R1/1 grid, both sides of which had been newly subjected to glow discharging in a stand-by time of 10 seconds and a blotting time of 4 seconds under 100% humidity conditions, with Vitrobot Mark at 4° C. The grid was subjected to plunge freezing in liquid ethane cooled at the liquid nitrogen temperature.

Cryo-electron microscopy (hereinafter, also referred to as cryo-EM) data were collected using a Titan Krios G3i microscope operating at 300 kV and equipped with a Gatan Quantum-LS energy filter (GIF) in electron counting mode and a Gatan K3 Summit direct electron detector. Each movie was recorded at a nominal magnification of 105,000-fold, corresponding to a calibrated pixel size of 0.83 Å, with an electronic exposure for 2.6 seconds at 15.8 e−/pix/sec and a cumulative exposure of 48.7 e−/A2. Data were acquired automatically in a defocus range of −0.8 to −1.6 mm according to an image shift method using SerialEM software, and 2,848 movies were acquired.

Dose-splitted movies were subjected to beam-induced motion correction and dose weighting using a MotionCor2 algorithm mounted in RELION-3, and the contrast transfer function (CTF) parameter was estimated using CTFFIND4.

Data processing was carried out using RELION-3. From the 2,848 images of motion-corrected and dose-weighted photomicrographic images, 1,960,343 particles were initially selected and extracted at a pixel size of 3.28 Å. These particles were subjected to several rounds of 2D and 3D classification. Next, the selected 143,063 particles were re-extracted at a pixel size of 1.05 Å and subjected to 3D refinement, per-particle defocus refinement, beam tilt refinement, Bayesian polishing, and 3D classification. The selected 87,253 particles were subjected to 3D refinement and subsequent post-processing of the maps, whereby the global resolution was improved to 3.3 Å according to the Fourier shell correlation (FSC)=0.143 criterion. Local resolution was estimated by RELION-3.

A model was manually built using COOT, and a protein model was reconstructed using Rosetta with respect to the density map. The model was refined using phenix.real_space_refine ver 1.16 and REFMAC 5.8 with secondary structure and base pair/stacking constraints. Structural verification was carried out using a PHENIX package, MolProbity. Curves showing the model and the full map were calculated using phenix. mtriage based on the final model and the full filtered sharp map. A cryo-EM density map was calculated using a UCSF chimera and molecular graphics. Figures were created with CueMol.

### Example 2

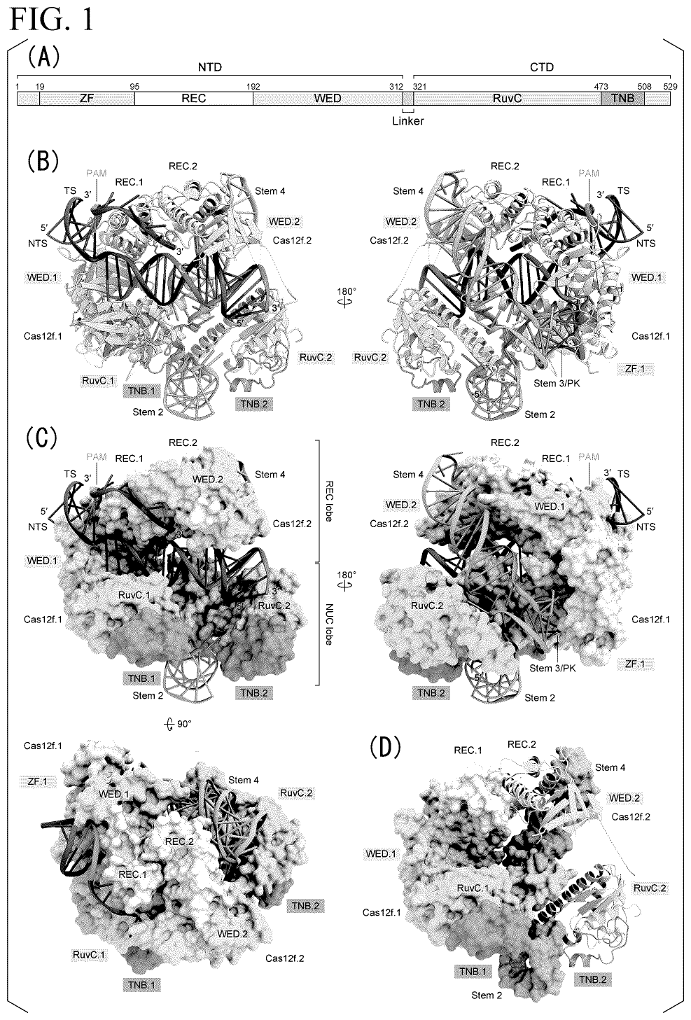

In order to reveal the DNA cleavage mechanism mediated by Cas12f, a cryoEM structure of a complex of Cas12f (D326A inactive mutant) and a target dsDNA (40 bp) having an sgRNA (180 nt) and TTTG PAM was determined at an overall resolution of 3.3 Å in Example 1 (see FIGS. 1A to D).

This structural analysis revealed that two Cas12f molecules (referred to as Cas12f.1 and Cas12f.2) aggregate with one sgRNA molecule to form a ribonucleoprotein effector complex (see FIGS. 1A to 1D).

Cas12f can be divided into an amino-terminal domain (NTD) and a carboxy-terminal domain (CTD), which are connected by a linker loop. NTD is composed of three domains of a wedge (WED) domain, a recognition (REC) domain, and a zinc finger (ZF) domain.

CTD is composed of a RuvC domain and another ZF domain called a target nucleic acid binding (TNB) domain. The Cas12f dimer adopts a bilobed architecture composed of a REC lobe and a nuclease (NUC) lobe, where a guide RNA-target DNA heteroduplex binds to a central channel between the two lobes (see FIGS. 1B and 1C). The REC lobe is formed from WED domains of Cas12f.1 (WED.1/ZF.1/REC.1) and Cas12f.2 (WED.2/ZF.2/REC.2), a ZF domain, and a REC domain. The NUC lobe is formed from RuvC domains of Cas12f.1 (RuvC.1/TNB.1) and Cas12f.2 (RuvC.2/TNB.2) and a TNB domain.

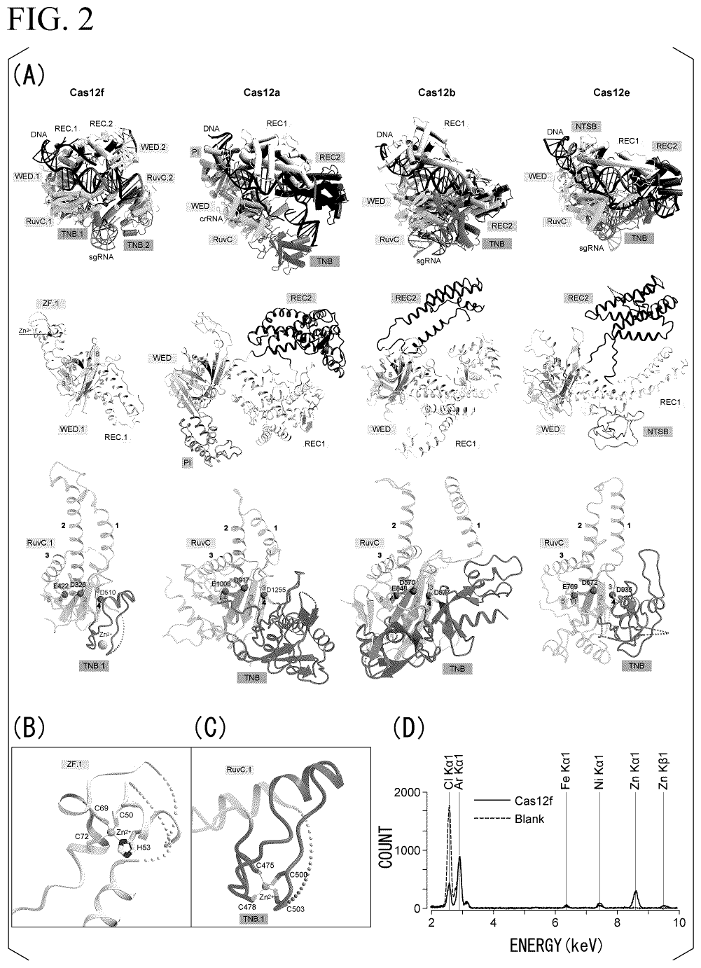

The WED domain contains a seven-stranded β-barrel adjacent to an α-helix and a β-hairpin, and the sequence similarity thereof is limited; however, it adopts an oligonucleotide/oligosaccharide binding (OB) fold similar to those of other Cas12 enzymes. The ZF domain and the REC domain are inserted between strands B1 and B2 of the WED domain. The ZF domain includes a CCCH-type ZF, where zinc ions are coordinated by C50, H53, C69, and C72 (see FIGS. 2A and 2B). The REC domain is composed of four helices and is much smaller than other Cas12 enzymes, and thus it mainly contributes to the compactness of Cas12f (see FIG. 2A). The RuvC domain has an RNase H fold and is composed of a five-stranded mixed β-sheet adjacent to four α-helices, where D326, E422, and D510 form a catalytic center similar to those of other Cas12 enzymes (see FIG. 2A). The TNB domain is inserted between the strand β5 and the helix α6 of the RuvC domain and contains a CCCC-type ZF, where zinc ions are coordinated by C475, C478, C500, and C503 (see FIGS. 2A and 2C). Four cysteine residues are conserved among Cas12f enzymes. The X-ray fluorescence elemental analysis of the purified Cas12f protein showed that Cas12f binds to zinc ions (see FIG. 2D).

The TNB domains of the Cas12 enzymes (also known as the Nuc domains of Cas12a and Cas12b and the target strand loading [TSL] domain of Cas12e) adopt structures different from each other (see FIG. 2A). Although the TNB domains of Cas12f and Cas12e contain two CXXC ZF motifs, the TNB domain of Cas12f is smaller than that of Cas12e. The TNB domains of Cas12a and Cas12b have unrelated structures and accelerate the cleavage of a target DNA by the RuvC domain. These domains adjacent to the RuvC domain are probably involved in the positioning of both the target strand (TS) and the non-target strand (NTS) in the RuvC active site. Therefore, in the present invention, these domains are collectively referred to as the TNB domain.

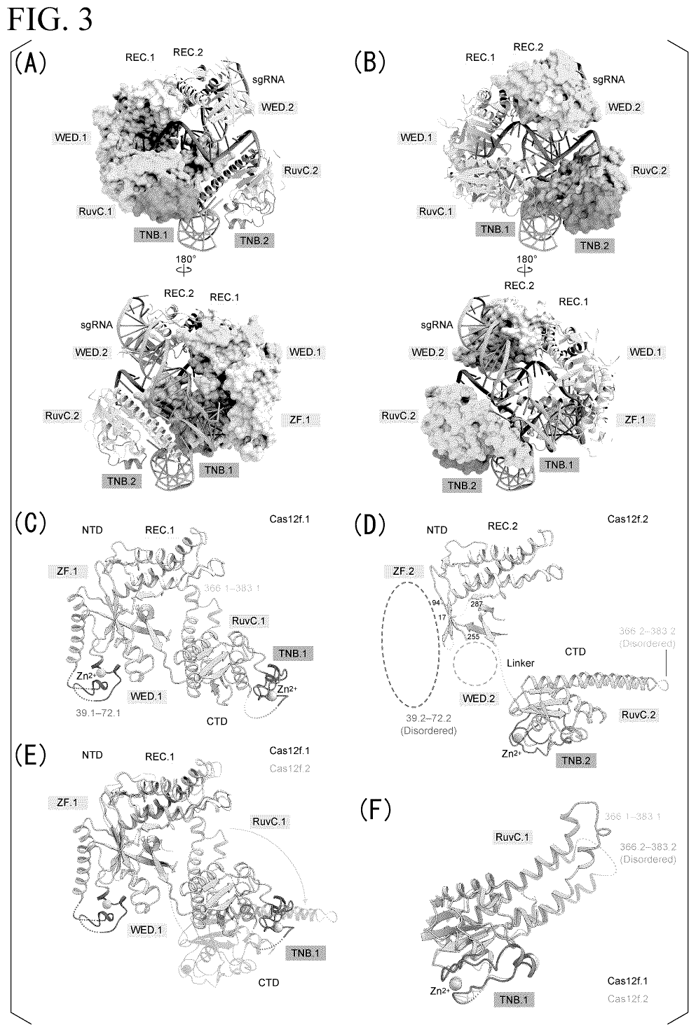

The structural comparison between Cas12f.1 and Cas12f.2 revealed significant differences in the arrangement of NTD and CTD. This is accelerated by a flexible linker loop and local structural changes in individual domains (see FIGS. 3A to 3F). Although ZF.1 (residues 18 to 93), WED.1 (residues 256 to 286), and RuvC.1 (residues 368 to 382) of Cas12f.1 interact with the guide RNA backbone, the equivalent regions of Cas12f.2 are exposed to a solvent and disordered in the complex structure (see FIGS. 1B to 1D and FIGS. 3A to 3F). This indicates that Cas12f undergoes a structural change upon binding to a guide RNA.

An sgRNA (U [−160] to C20) is composed of a 20-nt guide segment and a 160-nt RNA backbone and composed of five stems (stems 1 to 5) and a pseudoknot (PK) (FIGS. 4A and 4B and FIGS. 5A and 5B). The stem 1 (U [−160] to A [−141]), the upper stem region of the stem 2 (A [−129] to U [−103]), and the stem 5 (A [−29] to G [−13]) are disordered in structure, which suggests the flexibility of these regions. This structure reveals an unexpected feature of the guide RNA backbone. First, U (−84) to U (−79) form base pairs with A (−7) to A (−2), thereby forming PK (crRNA repeat-tracrRNA anti-repeat duplex 1 [R:AR-1]). PK stacks coaxially with the stem 3 to form a continuous helix. Second, G (−13) to A (−11) do not form base pairs with the previously predicted C (−26) to U (−28). It is shown that A (−12), A (−11), and G (−10) are flipped out of the stem, A (−29) to C (−26) instead of A (−25) to U (−22) form base pairs with U (−14) to G (−17), thereby completing the stem 5 (R:AR-2).

Using a complex of Cas12f and WT sgRNA (SEQ ID NO: 3: 5′-(GG)UUCACUGAUAAAGUGGAGAACCGCUUCACCAAAAGCUGUCCCUUAGG GGAUUAGAACUUGAGUGAAGGUGGGCUGCUUGCAUCAGCCUAAUGUCGAG AAGUGCUUUCUUCGGAAAGUAACCCUCGAAACAAAUUCAUUUGAAAGAAU

GAAGGAAUGCAACGGAAAUUAGGUGCGCUUGGC-3′) or a ΔAUUU mutant, obtained by deleting A (−25) to U (−22)), (SEQ ID NO: 4: 5′-(GG) UUCACUGAUAAAGUGGAGAACCGCUUCACCAAAAGCUGUCCCUUAGG GGAUUAGAACUUGAGUGAAGGUGGGCUGCUUGCAUCAGCCUAAUGUCGAG AAGUGCUUUCUUCGGAAAGUAACCCUCGAAACAAAUUCGAAAGAAUGAAG GAAUGCAACGGAAAUUAGGUGCGCUUGGC-3′), in vitro DNA cleavage activity was examined.

The Cas12f-sgRNA complex (500 nM) was prepared by mixing the purified Cas12f (1 mM) and an sgRNA (1 mM) at 50° C. for 3 minutes in 10 mL of a buffer F (5 mM Tris-HCl, pH 7.5, 25 mM NaCl, 5 mM MgCl2, and 1 mM DTT). The prepared Cas12f-sgRNA complex (2 mL, 500 nM, final concentration: 100 nM) was mixed with a linearized plasmid target (8 mL, 100 ng, final concentration: 5 nM) containing a target sequence of 20 bases and TTTG PAM and incubated at 50° C. in 10 mL of a reaction buffer (5 mM Tris-HCl, pH 7.5, 25 to 150 mM NaCl, 5 mM MgCl2, and 1 mM DTT). A quenching buffer containing EDTA (final concentration: 20 mM) and proteinase K (40 ng) was added thereto to stop the reaction. Aliquots (2 mL) were collected at 0.5, 1, 2, and 5 minutes and mixed with the quenching buffer (6 mL). Next, the reaction products were analyzed using a MultiNA Microchip Electrophoresis System (SHIMADZU CORPORATION). In order to determine the Cas12f DNA cleavage site, the linearized plasmid target (5 nM) was incubated with the Cas12f-sgRNA complex (100 nM) in the buffer F (50 mL) at 50° C. for 10 minutes. The reaction mixture was combined with the quenching buffer, followed by purification with Wizard SV Gel and PCR Clean-Up System. The purified cleavage product was analyzed by DNA sequencing (Eurofins Genomics LLC). In vitro cleavage experiments were carried out at least three times.

As shown in FIG. 5C, actually, the deletion of A (−25) to U (−22) did not affect the Cas12f-mediated DNA cleavage (see FIG. 5C).

Third, the sgRNA includes three base triples of G (−89) to C (−75)· A (−33) (the stem 3), G (−64) to C (−39)· A (−62) (the stem 4a), and U (−60) to A (−42)· A (−43) (the stem 4b). These stabilize the RNA backbone structure (see FIG. 5D).

Cas12f asymmetrically dimerizes through two interfaces (see FIG. 6A). The primary interface is symmetrical and is formed from the hydrophobic residues I118, Y122, I126, and M178 of REC.1 and REC.2 (see FIG. 5B). The secondary interface is asymmetric and is formed from the α1-α2 loop of RuvC. 1 and helices α1 and α2 of

RuvC.2 (see FIG. 5C). H371.1 and N369.1 each form hydrogen bonds with C405.2/D409.2 and R402.2, and L365.1 interacts with S347.2, N349.2, and D350.2.

Mutations of I118R, Y122A, I126R, and M178R reduced the DNA cleavage activity (see FIG. 6D).

Furthermore, the I118R/Y122A/I126R/M178R (RARR) mutant lacked the DNA cleavage activity (see FIG. 6D). In the presence of sgRNA, the wild-type (WT) Cas12f and the RARR mutant similarly eluted from the size exclusion column at a position corresponding to 198 kDa, consistent with the molecular weight of the (Cas12f) 2-sgRNA complex (184 kDa) rather than the molecular weight of the Cas12f-sgRNA complex (121 kDa) (see FIG. 7C).

These data showed that both the WT Cas12f and the RARR mutant form a dimer bound to the sgRNA, at least under the conditions tested.

In addition, size exclusion chromatography was used to analyze the oligomeric states of the WT Cas12f and the RARR mutant. As a control, a dimer mutant (Dimer) in which two Cas12f molecules are connected through a linker was generated (see FIG. 7D).

In the case where the sgRNA is not present, the WT and the RARR mutant were eluted from the column later as compared with the dimer mutant (see FIG. 7D), which suggests that the dimerization of Cas12f requires a guide RNA.

The sgRNA is widely recognized by both Cas12f.1 and Cas12f.2, and the helices α1 and α2 of RuvC. 1 and RuvC.2 play a central role in the RNA backbone recognition (FIG. 8 and FIGS. 9A to 9F). The stem 2 is recognized by RuvC.2 primarily through the interaction between the lower stem region thereof and the helix 1 of RuvC.2 (see FIG. 9C). The C (−140) to G (−91) base pairs in the basal region of the stem 2 stack with F359.2 and A360.2. G (−138) and A (−100) are sandwiched between K330.2 and F352.2 and each form hydrogen bonds with D348.2/R438.2 and K330.2. The deletion of the stems 1 and 2 (U [−160] to G [−94]) but not the stem 1 (U [−160] to A [−144]) reduced the Cas12f-mediated DNA cleavage (see FIG. 9G). From this, the functional importance of the stem 2 was confirmed.

The stem 3-PK helix is recognized by WED. 1, ZF. 1, and RuvC. 1 primarily through the interaction with the sugar phosphate backbone (see FIGS. 8, 9A, and 9B). In addition, the first U (−84) to A (−2) base pair of PK is recognized by N262.1 and K398.1 (see FIG. 9D). C (−1) between PK and the guide segment is strictly recognized by R259.1, T271.1, and E272.1. The first U (−92). U (−73) base pair in the stem 3 stacks with A360.2, R361.2, and 1364.2 of RuvC.2 (see FIG. 9C).

The stem 4 interacts with RuvC. 1 and REC.2, which bridges the REC and NUC lobe (FIGS. 8, 9A, and 9B). The lower stem region of the stem 4 (the stem 4a) is recognized by the α1-α2 loop of RuvC. 1 (see FIG. 9E). The bases C (−39), G (−66) to C (−37), and A (−35) of the stem 4a each form hydrogen bonds with G375.1, H376.1, and K383.1 of the α1-α2 loop. In particular, C (−40) is flipped out of the stem 4 and extensively interacts with the side chains of A378.1, K381.1, and L382.1 and the main chains of K367.1, G375.1, and G377.1 (see FIG. 9E). As a result, C (−40) is involved in RNA-DNA heteroduplex recognition. The deletion of the α1-α2 loop (residues 366 to 383) abolishes the DNA cleavage activity (see FIG. 9G). On the other hand, the equivalent region of Cas12f.2 is exposed to a solvent and disordered in the complex structure (see FIG. 7A). These results show that the interaction between RuvC. 1 and the stem 4 is important for Cas12f-mediated DNA cleavage. The upper stem regions of the stem 4 (the stems 4b and 4c) interact with REC.2 through charge and shape complementarity (see FIG. 9B).

The stem 5 is recognized by WED.1, ZF.1, and REC.1 (FIGS. 8, 9A, and 9B). A (−12), A (−11), and G (−10) are flipped out of the stem and are each sandwiched by W95.1/K299.1, Y82.1/W95.1, and V15.1/L253.1/Q257.1 (see FIG. 9F). In addition, G (−10) adopts the syn conformation and forms a plurality of hydrogen bonds with D213.1, S255.1, and T256.1. Although the deletion of the ZF motif (residues 39 to 72) of the ZF domain did not impair the DNA cleavage activity (see FIG. 9G), ZF.2 was structurally disordered (see FIG. 7A), which showed the functional importance of the interaction between ZF.1 and the guide RNA.

The guide RNA-target DNA heteroduplex is housed within the positively charged central channel and recognized through the interaction with the sugar-phosphate backbone thereof (FIGS. 8 and 9B), which explains the RNA-dependent DNA recognition mechanism of Cas12f.

The seven nucleotides (dG1* to dT7*) of the single-stranded NTS are recognized by REC.1 and REC.2/WED.2 by a sequence-independent method.

H139.1, 1131.1/Y232.2, and P234.2 each form stacking interactions with the dG1*, dA3*, and dA5* bases of NTS, and N133.1, K173.1, R103.2, and R292.2 interact with the sugar-phosphate backbone (see FIG. 9H).

No distinct densities of the nucleotides (−8) to (−1) and 28 to 32 of TS and the nucleotides (−12*) to (−8*) and 8* to 28* of NTS are observed, which suggests the flexibility thereof.

The duplex containing the TTTG PAM is recognized by REC. 1 and WED.1 (see FIG. 9I). The dT (−4*) to dT (−2*) bases of the PAM form hydrophobic interactions with A156.1 and Y146.1. Y146.1 also interacts with the main chain phosphate group between dT (−4*) and dT (−3*). The dG (−1*) base forms a hydrogen bond and a stacking interaction with each of S142.1 and R163.1. In addition, the bases of dA24 and dA23, which form base pairs with dT (−4*) and dT (−3*), each form hydrogen bonds with Y202.1 and Q197.1. Although the Y146A and Q197A mutations each abolished and reduced the DNA cleavage activity (FIG. 9G), the equivalent residues of Cas12f.2 were not in contact with the nucleic acid (see FIG. 7A), from which the functional importance of Y146.1 and Q197.1 in PAM recognition was confirmed. Collectively, these results explain the TTTR PAM directivity of Cas12f. The phosphate backbone between dC21 and dC20 of NTS is recognized by K198.1 and S286.1 of WED.1 (K198.2 and S286.2 are disordered) (see FIGS. 91 and 7A), whereby the heteroduplex formation is accelerated.

The K198A and S286A mutants each exhibit substantially and slightly reduced cleavage activity (see FIG. 9G), which suggests the important role of K198.1 on DNA unwinding.

From the determination of the sequence of the target DNA cleavage product as shown in FIG. 10A, Cas12f cleaves each of TS and NTS at 24 nt and 22nt upstream of PAM (see FIG. 10A). The Cas12 enzyme generally cleaves both TS and NTS in a single RuvC active site, and the TNB (also called Nuc or TSL) domain accelerates the loading of TS and NTS into the RuvC active site.

In the Cas12f structure, the location of RuvC. 1 is similar to that of the RuvC domains of other Cas12 enzymes; however, RuvC.2 is closer to the 5′ terminal of TS (see FIG. 10B). In order to investigate the Cas12f-mediated DNA cleavage mechanism, mutants of D326.1A and D326.2A, in which RuvC. 1 and RuvC.2 were selectively inactivated, were prepared (see FIG. 10C). Since the dimer mutant (see FIG. 7A) can bind to an sgRNA in two different orientations, the two RuvC domains thereof (the N-terminal RuvC.1 and the C-terminal RuvC.2) can be arranged at both positions of RuvC.1 and RuvC.2 in the Cas12f-sgRNA-target DNA complex. The residues 366 to 383 of RuvC. 1 are involved in RNA backbone recognition and important for DNA cleavage (see FIGS. 9E and 9G), whereas the residues 366 to 383 of RuvC.2 are exposed to a solvent and disordered in the complex structure (see FIG. 7A). In order to selectively inactivate the two RuvC active sites, L365.2 and P384.2 of the Dimer mutant were connected with a linker, and the residues 366 to 383 of RuvC.2 were deleted to prepare a DimerA mutant (see FIG. 10C). As expected, the Dimer and DimerA mutants exhibited comparable activity, although the efficiency was low as compared with the WT Cas12f (see FIG. 10D). These results showed that the DimerA mutant can function only in the case where it binds to an sgRNA in a defined orientation, in which the N-terminal RuvC.1 and C-terminal RuvC.2 are at positions of RuvC. 1 and RuvC.2. Next, D326.1 and D326.2 of the DimerA mutant were substituted with alanine to prepare each of D326.1A and D326.2A mutants (see FIG. 10C). Since the D326.1A and D326.2A mutants can function only in the case of binding to an sgRNA in a defined orientation, RuvC.1 and RuvC.2 are each selectively inactivated the D326.1A and D326.2A mutants. In particular, the D326.1A mutant lacked the DNA cleavage activity, whereas the D326.2A mutant exhibited activity comparable to that of the DimerA mutant (see FIG. 10D), which suggested that RuvC. 1 cuts both TS and NTS.

From the structural comparison with Cas12e, it was suggested that F487.1 of TNB.1 interacts with the target DNA (see FIG. 10E). Actually, the F487A mutant exhibited reduced activity (see FIG. 10D), and TNB. 1 was involved in DNA binding, which suggested that, like other Cas12 enzymes, F487 accelerates the recruitment of TS to RuvC.1.

### Example 3

5×104 HEK293 cells were seeded in each well in a 48-well plate. The next day, HEK293 cells were transfected with a plasmid (200 ng) containing each of genes encoding mutant Cas12f (I118C, Y122C, N133R, E174R, N177R, S187R, N470R, and N483R) and a sgRNA plasmid (SEQ ID NO: 5; 150 ng). Genomic DNA was extracted from the cells recovered 48 hours after transfection, PCR was carried out, and Indel frequency was analyzed using MaltiNA. The results are shown in FIG. 11. In FIG. 11, WT-unCas12 indicates wild-type Cas12f, and unCas12 (1) to (8) indicate I118C, Y122C, N133R, E174R, N177R, S187R, N470R, and N483R in order. As shown in FIG. 11, it was confirmed that each mutant has an increased enzymatic activity.

### INDUSTRIAL APPLICABILITY

According to the present invention, it is possible to provide an engineered Cas12f protein that is capable of being used as a genome editing tool.

1. A ribonucleoprotein effector complex that consists of: (i) a guide RNA and (ii) a protein comprising a sequence having 80% homology to SEQ ID NO: 1 including any one of (a) to (c):
(a) at least one substitution of an amino acid residue selected from the group consisting of I118, Y122, I126, and M178,
(b) one to several amino acids are deleted, inserted, substituted, or added in a portion other than amino acid positions 118, 122, 126, and 178 relative to SEQ ID NO:1,
(c) an amino acid sequence having 95% or more identity in a portion other than the amino acid positions 118, 122, 126, and 178 of the amino acid sequence represented by (a) above,
wherein the protein forms a homodimer and forms the ribonucleoprotein effector complex with the guide RNA.

2. The ribonucleoprotein effector complex according to claim 1,
wherein the substitution of the amino acid residue in the amino acid sequence represented by (a) above is a substitution with cysteine.

3. The ribonuceloprotein effector complex according to claim 1,
wherein the substitution of the amino acid residue in the amino acid sequence represented by (a) above is I118C and/or Y122C.

4. The ribonucleoprotein effector complex according to claim 1,
further comprising a substitution of an amino acid residue of A156 and/or Y146.

5. The ribonuceloprotein effector complex according to claim 4,
wherein in the amino acid sequences (a) to (c) further comprising an amino acid substitution of A156N.

6. A ribonucleoprotein effector complex that consists of: (i) a guide RNA and (ii) a protein comprising a sequence having 80% homology to SEQ ID NO: 1 including any one of (a) to (c):
(a) a substitution of an amino acid residue of A156 and/or Y146,
(b) one to several amino acids are deleted, inserted, substituted, or added in a portion other than amino acid positions 156 and 146 relative to SEQ ID NO: 1,
(c) an amino acid sequence having 95% or more identity in a portion other than the amino acid positions 156 and 146 relative to SEQ ID NO: 1.

7. The ribonucleoprotein effector complex according to claim 6, further comprising a
substitution of the amino acid residue A156N.

8. The ribonucleoprotein effector complex according to claim 1, further comprising at least one mutation selected from the group consisting of N133R, E174R, N177R, S187R, N470R, and N483R.

9. A polynucleotide encoding the protein according to claim 1.

10. A vector comprising the polynucleotide according to claim 9.

11. A composition comprising:
the protein according to claim 1 and
a single guide RNA.

12. A method for editing genome in a cell using the composition according to claim 11.

13. A method for site-specifically modifying a target double-stranded polynucleotide in a cell, the method comprising:
bringing a target double-stranded polynucleotide, the protein according to claim 1, and a guide RNA into contact with each other,
wherein the protein cleaves the target double-stranded polynucleotide at a cleavage site located upstream of a PAM sequence in the target double-stranded polynucleotide, and
the protein modifies the target double-stranded polynucleotide in a region that is determined by complementary binding of the guide RNA and the target double-stranded polynucleotide.

14. A method for site-specifically modifying a target double-stranded polynucleotide in an isolated cell, the method comprising:
bringing a target double-stranded polynucleotide, a complex of the protein according to claim 1 and a nucleic acid base converting enzyme, and a guide RNA into contact with each other,
wherein the protein specifically binds to the target double-stranded polynucleotide through the guide RNA, where the protein does not cleave the target double-stranded polynucleotide or cleaves only one strand of the target double-stranded polynucleotide, and
the protein modifies the target double-stranded polynucleotide in a region that is determined by complementary binding of the guide RNA and the target double-stranded polynucleotide.

15. A method for regulating expression of a gene in an isolated cell, the method comprising:
bringing a target double-stranded polynucleotide associated with the gene, the protein according to claim 1, a guide RNA, and an effector molecule into contact with each other,
wherein the protein lacks an ability to cleave one or both strands of a target double-stranded polynucleotide, and
the protein specifically binds to the target double-stranded polynucleotide through the guide RNA, and consequently, the effector molecule specifically acts on the target double-stranded polynucleotide to regulate expression of the gene.

16. A composition comprising:
the polynucleotide according to claim 9 and
a guide RNA.

17. A method for editing genome in an isolated cell using the composition according to claim 16.

18. A composition comprising:
the vector according to claim 10 and
a guide RNA.

19. A method for editing genome in an isolated cell using the composition according to claim 18.
